(19) United States
(12) Patent Application Publication
(10) Pub. No.: US 2019/0002819 A1
(43) Pub. Date: Jan. 3, 2019

(54) HEAT PRIMING OF BACTERIAL SPORES

(71) Applicant: NOVOZYMES BIOAG A/S, Bagsvaerd (DK)

(72) Inventor: Jared Heffron, Salem, VA (US)

(73) Assignee: NOVOZYMES BIOAG A/S, Bagsvaerd (DK)

(21) Appl. No.: 16/064,328

(22) PCT Filed: Dec. 27, 2016
(86) PCT No.: PCT/US2016/068642

§ 371 Date: Jun. 20, 2018

Related U.S. Application Data
(60) Provisional application No. 62/271,577, filed on Dec. 28, 2015.

(51) Int. Cl. C12N 1/20; A23K 10/18; A23K 50/80; A01N 63/00

(52) U.S. Cl. CPC C12N 1/20; A23K 10/18; C12N 3/00; A01N 63/00; A23K 50/80

(57) ABSTRACT

The present invention relates to methods for heat treating spores, which improves subsequent germination properties.

### FIELD OF THE INVENTION

The present invention relates to methods for heat treating spores, which improves subsequent germination properties, and compositions containing the treated spores.

### BACKGROUND

Bacterial spores are not part of a sexual cycle but are resistant structures used for survival under unfavorable conditions. When using commercial products based on bacterial spores, the endospore germinates to a vegetative state to carry-out metabolism and facilitate a desired action for product efficacy. It is well documented that germination of a population of bacterial spores is highly heterogeneous. Consequently, a spore population is likely to germinate over a relatively large span of time; in natura some spores may require weeks to months of incubation before germination begins. Furthermore, a measurable contingent of the spores may not germinate at all during application. Thus the efficacy of bacterial spore-based products can be significantly improved by making germination occur more homogenously.

### SUMMARY OF THE INVENTION

In a first aspect, the present invention provides a stabilized bacterial spore composition comprising:

(a) a carrier;
(b) optionally one or more germinants; and
(c) a bacterial spore population which has been treated with a sub-lethal heat treatment at 50-80° C. for more than 30 minutes followed by cooling to below 30° C.;
wherein the bacterial spore population exhibits improved germination after 24 hours compared to a non-treated, but otherwise identical, bacterial spore population.

The invention further provides a method for preparing a stabilized bacterial spore composition comprising the steps of:

(a) treating a bacterial spore population with a sub-lethal heat treatment at 50-80° C. for more than 30 minutes followed by cooling to below 30° C.;
(b) mixing the treated bacterial spore population with a carrier, and optionally one or more germinants; and
(c) storing the bacterial spore population for at least 24 hours before or after step (b); wherein the bacterial spore population exhibits improved germination after 24 hours compared to a bacterial spore population which did not receive the treatment in (a).

Other aspects and embodiments of the invention are apparent from the description and examples.

### BRIEF DESCRIPTION OF THE DRAWINGS

FIG. 1. An example of kinetic germination data generated for this work. (A) A curve demonstrating the germination kinetics of a population of spores as determined by the release of DPA over time. T_{lag} and Gₘₐₓ are indicated by arrows. (B) The dotted line sample demonstrates faster initiation (shorter T_{lag}) and improved efficiency (greater Gₘₐₓ) of germination compared to the solid line sample. (C) The dotted line sample demonstrates greater efficiency (Gₘₐₓ), but not faster initiation (T_{lag}) of germination compared to the solid line sample.

FIG. 2. B. subtilis spore germination kinetics after heat priming is dose dependent on both temperature and duration of the treatment. Spores were heat primed at 65° C. (FIG. 2A), 70° C. (FIG. 2B), and 75° C. (FIG. 2C) for 30, 120, and 240 m, as indicated, and stored at 4° C. for 1 day before these assays. A negative control received no heat priming and is present in each chart for comparison purposes. The T_{lag} shortened and the Gₘₐₓ increased as the priming temperature increased (compare the same time of heat treatment between the panels) and as the duration of priming increased (compare increasing times of heat treatment within panels). All treatments were significantly different from the control p>0.001 as determined by a Tukey's HSD comparison of the means for each time point. Shown are the means of three independent replicates for each group. Similar results were obtained from the spores when tested 4, 7, 14, 30 and 60 days after priming. Error bars are omitted for clarity

FIG. 3. B. pumilus spore germination kinetics after heat priming was maximized at 60° C. Spores were heat primed for 60 m at 60° C., 65° C., 68° C., and 70° C. (as indicated) and stored at 4° C. for sixteen days before these assays. A negative control received no heat priming and is present in the chart for comparison purposes. The T_{lag} shortened and the Gₘₐₓ significantly increased compared to the control when primed at 60° C.-68° C. (p>0.001 as determined by a Tukey's HSD). Priming at 70° C. led to a germination curve with a Gₘₐₓ that was not significantly different from the control. Shown are the means of three independent replicates for each group. Similar results were obtained from the spores when tested 0, 1, 2, 7, and 33 days after priming. Error bars are omitted for clarity.

FIG. 4. Shrimp feed coated with SB3281 and/or MF1048 spores improve survival of shrimp under EMS challenge. The study that yielded the data in FIG. 4 is described in Example 3.

FIG. 5. Shrimp Survival after EMS Disease Treatment. Feed was coated with spores of SB3281 (indicated as SB3281NA), MF1048 (MF1048NA), or no spores. One set of both SB3281 and MF1048 were heat activated at 65° C. 30 m prior to coating on feed (3281A and 1048A, respectively). After 7 days of feeding, the shrimp received a lethal dose of EMS except for one control treatment (Control (−)). The percentage of surviving shrimp (y-axis) was assessed regularly for 104 h post-infection.

### DETAILED DESCRIPTION

A non-limiting example of spore-forming bacteria is the Bacillus species. In their non-spore form, they are a typical eating, growing, dividing cell; they are typically referred to as vegetative. In the spore state they have no measurable metabolism, but are one of the most durable biological structures known to man. Consequently, when a species of Bacillus carries out a useful function it is in a vegetative form, because that is when it is enzymatically active. But to be produced, packaged, and stockpiled for industrial needs the spore form is preferred, because of its extreme hardiness. Ultimately, a Bacillus based product is made and sold as a spore, but when applied by the end-user the spore must transition into the vegetative state that is capable of performing the desired function. This process of a spore becoming a vegetative cell is called germination.

Bacillus has evolved to in such a manner as to allow individuals to have markedly different requirements for germination. That means that “spore A” may require L-alanine at a high concentration plus a small amount of d-glucose in order to be convinced to germinate, but another individual, “spore B”, genetically identical to “A”, will only respond if the d-glucose concentration is high with almost no requirement for L-alanine. The anthropomorphized argument explaining this phenomenon is that the spores do not want to “put all their eggs in one basket”. For example, if the first spore germinates due to high L-alanine in an environment that has low pH, then it will die. But spore B, which did not germinate, remains dormant, survives, and waits for its preferred conditions where the pH may have neutralized. Thus it is almost assured that among a logarithmic number of spores several individuals manage to germinate in perfect conditions for growth and succeed in perpetuating the species' gene pool.

In nature it behooves Bacillus to have variability in germination requirements, but in industrial microbiology it creates a problem. A typical product consists of a batch of dormant spores as the main ingredient. They are stable during formulation, packaging, and shelf storage, but upon application the vegetative form is needed to carry out enzymatic activity and metabolism to perform the desired function. The heterogeneity of germination requirements results in treatments where less than 100% of the main ingredient becomes active. In many cases less than 50% will germinate. Thus any means that makes the requirements for spore germination more homogenous can improve the efficacy of the product.

Sub-lethal treatment at temperatures in excess of 37° C. for a particular duration of time synchronizes the germination requirements for a population of spores. The exact mechanism has remained a mystery for well over 50 years. Regardless of the mechanism, the result is that more spores will respond to a germinant signal in a shorter period of time, and in some cases the requirements for germination will be reduced. Thus an invention that harnesses the activation/priming/synchronization abilities of heat can result in a spore-based product that initiates faster, in a wider range of treatment environments, and at higher efficiency. The benefits are manifold.

The present invention provides an advantageous method for heat treating spores, which permits a bacterial spore preparation to be prepared in a shelf-stable form that is capable of demonstrating improved germination kinetics upon application.

In theory, any product that requires bacterial spores to germinate before being efficacious will be more efficient if germination is improved. Any product that uses bacterial spores as an active agent can demonstrate improved function and potency if they germinate faster and at a higher efficiency.

Thus, the applications for this invention are diverse with any product where bacterial spores are an active ingredient. For bioagriculture, direct-fed microbials, waste water treatment, and cleaning applications the spores can be pre-treated by the manufacturer before being released to a customer. The germination of a treated population of bacterial spores will be more homogenous than when left to be triggered naturally and will improve the efficacy of the product.

In basic spore research the ability to make a population commit to germination with 90% efficiency has been essential to generating significant and reproducible data, because a non-heat activated batch may germinate with only 40-50% efficiency.

Typically, those practiced in the art of spore germination follow the heat treatment with a short (5-15 minutes) cooling at 4° C. This is performed to better prepare the sample for analysis at a relevant experimental temperature. Anecdotally, it is common for those practiced in the art to insist that spores are tested within 1 hour of cooling because they can reset back to a non-activated state where germination is no longer as efficient. Very little empirical work has been performed on the details of heat activation reversibility, but it has been demonstrated that the measurable changes caused by heat activation revert back to a non-heated state. The reversal is very noticeable in as little as 15 minutes of cooling. These results along with common practices in the spore research community support the argument that heat activation is beneficial only to spores in the short term. After prolonged cold storage (>1 hour) one would not expect to see a measurable benefit to spore germination rates regardless of whether they were ever heat activated.

### Definitions

In general, the terms and phrases used herein have their art-recognized meaning, which can be found by reference to standard texts, journal references, and context known to those skilled in the art. The following definitions are provided to clarify their specific use in context of the disclosure.

As used herein, the singular forms “a”, “an” and “the” are intended to include the plural forms as well, unless the context clearly indicates otherwise.

As used herein, the terms “aquaculture”, “aquaculturing”, “aquafarm”, and “aquafarming” can be used interchangeably and refer to the cultivation, breeding, raising, production, propagation and/or harvesting of an aquatic or marine animal, generally in an artificial environment such as a tank (e.g., an aquarium), a pond, a pool, a paddy, a lake, etc., or in an enclosed or fenced off portion of the animals natural habitat, such as a pond, a pool, a paddy, a lake, an estuary, an ocean, a marsh (e.g., a tidal marsh), a lagoon (e.g., a tidal lagoon), etc. As used herein, the term “mariculture” refers to aquaculture practiced in marine environments and in underwater habitats.

As used herein, the terms “aquatic animal”, “marine animal” or “aquatic and/or marine animals” refer to organisms that live in an aquatic or marine environment. Non-limiting examples include fish, e.g., osteichthyes (including, but not limited to catfish, tilapia, trout, salmon, perch, bass, tuna, wahoo, tuna, swordfish, marlin, grouper, sturgeon, snapper, eel and walleye) and chondrichthyes (including, but not limited to sharks, rays, and skates), crustaceans (including, but not limited to crabs, lobsters, crayfish, shrimp, krill, and prawn) and mollusks (including, but not limited to snails, slugs, conch, squid, octopus, cuttlefish, clams, oysters, scallops, and mussels).

As used herein, the term “agriculturally beneficial ingredient(s)” means any agent or combination of agents capable of causing or providing a beneficial and/or useful effect in agriculture.

As used herein, the term “carrier” means an “agronomically acceptable carrier.” An “agronomically acceptable carrier” means any material which can be used to deliver the actives (e.g., microorganisms described herein, germinants, agriculturally beneficial ingredient(s), biologically active ingredient(s), etc.) to a plant or a plant part (e.g., plant foliage), and preferably which carrier can be applied (to the plant, plant part (e.g., foliage, seed), or soil) without having an adverse effect on plant growth, soil structure, soil drainage or the like.

As used herein, the term “soil-compatible carrier” means any material which can be added to a soil without causing/having an adverse effect on plant growth, soil structure, soil drainage, or the like.

As used herein, the term “seed-compatible carrier” means any material which can be added to a seed without causing/having an adverse effect on the seed, the plant that grows from the seed, seed germination, or the like.

As used herein, the term “foliar-compatible carrier” means any material which can be added to a plant or plant part without causing/having an adverse effect on the plant, plant part, plant growth, plant health, or the like.

As used herein, the term “foliage” means all parts and organs of plants above the ground. Non-limiting examples include leaves, needles, stalks, stems, flowers, fruit bodies, fruits, etc. As used herein, the term “foliar application”, “foliarly applied”, and variations thereof, is intended to include application of an active ingredient to the foliage or above ground portions of the plant, (e.g., the leaves of the plant). Application may be effected by any means known in the art (e.g., spraying the active ingredient).

As used herein, the term “germinant(s)” means any substance or compound that induces microbial spore germination (e.g., a substance or compound that induces the germination of a microbial spore, such as a bacterial spore).

As used herein, the terms “plant(s)” and “plant part(s)” means all plants and plant populations such as desired and undesired wild plants or crop plants (including naturally occurring crop plants). Crop plants can be plants, which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material and vegetative and generative propagation material (e.g., cuttings, tubers, rhizomes, off-shoots and seeds, etc.).

### Heat Priming of Bacterial Spores

Bacterial spores are heat activated in an aqueous environment with high, but sub-lethal, heat for a set period of time. Typical temperature range is 50-80° C., preferably 60-75° C. and a duration of more than 30 minutes; preferably a duration of 30-240 minutes. The optimal heat treatment temperature and duration is species dependent, but are easily determined by following the procedures outlined in Example 2. Immediately after the heat treatment, the spores are cooled to below 30° C., preferably to room temperature (˜22° C.) before storage at typical temperatures used in storage facilities, such as 22° C. or 4° C. The germination properties of the heat treated spores do not change over time, and therefore the heat treated spores can be stored more or less indefinitely.

In some examples, a bacterial spore population may be treated with heat at or about a temperature of 50, 55, 60, 65, 70, 75, 80° C., or other temperatures. In some examples, the spores may be treated with heat at or about 50-60° C., 60-70° C., 70-80° C., 50-55° C., 55-60° C., 60-65° C., 65-70° C., 70-75° C., 75-80° C., or other temperature ranges. The duration of the heat treatment may be at or about 30, 60, 90, 120, 150, 180, 210, 240, or more minutes.

After heat treatment, the bacterial spores generally are cooled to below 30° C. In some examples, the temperature to which the spores are cooled may be 29, 28, 27, 26, 25, 22, 20, 15, 10, 5, 4° C., or other temperatures. In some examples, the spores may be cooled to a temperature range of less than 30° C. but 4° C. or greater. In some examples, the duration of the cooling is at least 24 h (1 day), 2 days, 3 days, 4 days, 5 days, 10 days, 15 days, 16 days, 20 days, or longer. In some examples, the duration of the cooling may be greater than 15 minutes. In some examples, bacterial spores that are cooled to below 30° C. may subsequently be stored at typical temperatures used in storage facilities, such as 22° C. or 4° C.

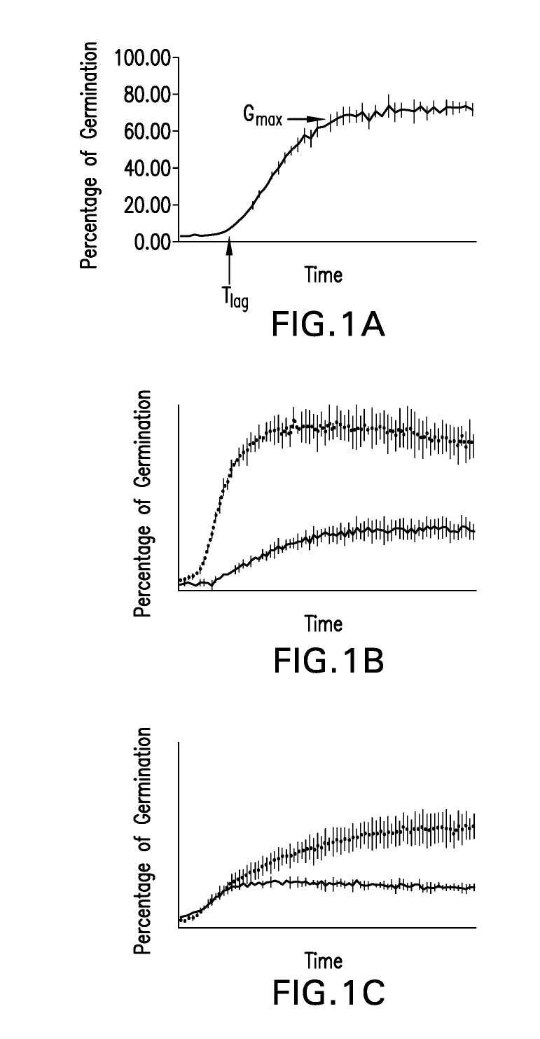

A population of bacterial spores that has been heat treated and cooled, as described herein, generally exhibit improved germination at or after the cooling process (generally cooling for at least 1 day) as compared to a substantially identical population of bacterial spores that has not been heat treated and cooled. In one example, the improved germination of the heat treated and cooled bacterial spores may be one or both of a decreased T_{lag} and increased Gₘₐₓ as compared to the bacterial spores that has not been heat treated and cooled (see FIG. 1). In some examples, the improved germination may be exhibited after 24, 48 or 72 h, 1, 2, 5, 7, 10, 15, 20, 30, 33, 45 or 60 days, 3, 4, 5, 6, 7 or 8 weeks, 1, 2, 4, 6, 8, 10 or 12 months, or 1 or more years. In some examples, the improved germination characteristics of the heat treated and cooled spores may last indefinitely. In some examples, the improved germination characteristics of the heat treated and cooled spores may last for at least 1, 2, 5, 7, 10, 15, 20, 30, 33, 45 or 60 days, 3, 4, 5, 6, 7 or 8 weeks, 1, 2, 4, 6, 8, 10 or 12 months, or 1, 2, 3, 4, 5, 10 or more years.

### Bacterial Spores

The spores used in the present invention are bacterial spores, such as endospores.

The one or more bacterial spores of the invention are derived from spore forming bacterial strains. Methods for producing stabilized microorganisms, and bacterial spores specifically, are known in the art. See for example, Donnellan, J. E., Nags, E. H., and Levinson, H. S. (1964) “Chemically defined, synthetic media for sporulation and for germination and growth of Bacillus subtilis”, Journal of Bacteriology, 87(2):332-336; and Chen, Z., Li, Q., Liu, H. Yu, N., Xie, T., Yang, M., Shen, P., Chen, X. (2010) “Greater enhancement of Bacillus subtilis spore yields in submerged cultures by optimization of medium composition through statistical experimental designs.”, Appl. Microbiol. Biotechnol., 85:1353-1360.

An example condition under which vegetative cells of bacteria form spores may be limiting amounts of nutrients needed for vegetative growth of the bacteria. Methods for obtaining bacterial spores from vegetative cells are well known in the field. In some examples, vegetative bacterial cells are grown in liquid medium. Beginning in the late logarithmic growth phase or early stationary growth phase, the bacteria may begin to sporulate. When the bacteria have finished sporulating, the spores may be obtained from the medium, by using centrifugation for example. Various methods may be used to kill or remove any remaining vegetative cells. Various methods may be used to purify the spores from cellular debris and/or other materials or substances. Bacterial spores may be differentiated from vegetative cells using a variety of techniques, like phase-contrast microscopy or tolerance to heat, for example.

Non-limiting examples of spore forming bacterial strains include strains from the genera Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Omithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, and/or Vulcanobacillus.

In a particular embodiment, the one or more spore forming bacteria is a bacteria selected from the genera consisting of Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Omithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, Vulcanobacillus, and combinations thereof.

In another embodiment, the one or more bacterial strains is a strain of Bacillus spp., e.g., Bacillus alcalophilus, Bacillus alvei, Bacillus aminovorans, Bacillus amyloliquefaciens, Bacillus aneurinolyticus, Bacillus aquaemaris, Bacillus atrophaeus, Bacillus boroniphilius, Bacillus brevis, Bacillus caldolyticus, Bacillus centrosporus, Bacillus cereus, Bacillus circulans, Bacillus coagulans, Bacillus firmus, Bacillus flavothermus, Bacillus fusiformis, Bacillus globigii, Bacillus infernus, Bacillus larvae, Bacillus laterosporus, Bacillus lentus, Bacillus licheniformis, Bacillus megaterium, Bacillus, mesentericus, Bacillus mucilaginosus, Bacillus mycoides, Bacillus natto, Bacillus pantothenticus, Bacillus polymyxa, Bacillus pseudoanthracis, Bacillus pumilus, Bacillus schlegelii, Bacillus sphaericus, Bacillus sporothermodurans, Bacillus stearothermophillus, Bacillus subtilis, Bacillus thermoglucosidasius, Bacillus thuringiensis, Bacillus vulgatis, Bacillus weihenstephanensis, and combinations thereof.

In another embodiment, the one or more bacterial strains is a strain of Brevibacillus spp., e.g., Brevibacillus brevis; Brevibacillus formosus; Brevibacillus laterosporus; or Brevibacillus parabrevis, and combinations thereof.

In another embodiment, the one or more bacterial strains is a strain of Paenibacillus spp., e.g., Paenibacillus alvei; Paenibacillus amylolyticus; Paenibacillus azotofixans; Paenibacillus cookii; Paenibacillus macerans; Paenibacillus polymyxa; or Paenibacillus validus, and combinations thereof.

In a more particular embodiment, the one or more bacterial strains is a strain of Bacillus selected from the group consisting of Bacillus pumilus strain NRRL B-50016; Bacillus amyloliquefaciens strain NRRL B-50017; Bacillus amyloliquefaciens strain PTA-7792 (previously classified as Bacillus atrophaeus); Bacillus amyloliquefaciens strain PTA-7543 (previously classified as Bacillus atrophaeus); Bacillus amyloliquefaciens strain NRRL B-50018; Bacillus amyloliquefaciens strain PTA-7541; Bacillus amyloliquefaciens strain PTA-7544; Bacillus amyloliquefaciens strain PTA-7545; Bacillus amyloliquefaciens strain PTA-7546; Bacillus subtilis strain PTA-7547; Bacillus amyloliquefaciens strain PTA-7549; Bacillus amyloliquefaciens strain PTA-7793; Bacillus amyloliquefaciens strain PTA-7790; Bacillus amyloliquefaciens strain PTA-7791; Bacillus subtilis strain NRRL B-50136 (also known as DA-33R, ATCC accession No. 55406); Bacillus amyloliquefaciens strain NRRL B-50141; Bacillus amyloliquefaciens strain NRRL B-50399; Bacillus licheniformis strain NRRL B-50014; Bacillus licheniformis strain NRRL B-50015; Bacillus amyloliquefaciens strain NRRL B-50607; Bacillus subtilis strain NRRL B-50147 (also known as 300R); Bacillus amyloliquefaciens strain NRRL B-50150; Bacillus amyloliquefaciens strain NRRL B-50154; Bacillus megaterium PTA-3142; Bacillus amyloliquefaciens strain ATCC accession No. 55405 (also known as 300); Bacillus amyloliquefaciens strain ATCC accession No. 55407 (also known as PMX); Bacillus pumilus NRRL B-50398 (also known as ATCC 700385, PMX-1, and NRRL B-50255); Bacillus cereus ATCC accession No. 700386; Bacillus thuringiensis ATCC accession No. 700387 (all of the above strains are available from Novozymes, Inc., USA); Bacillus amyloliquefaciens FZB24 (e.g., isolates NRRL B-50304 and NRRL B-50349 TAEGRO® from Novozymes), Bacillus subtilis (e.g., isolate NRRL B-21661 in RHAPSODY®, SERENADE® MAX and SERENADE® ASO from Bayer CropScience), Bacillus pumilus (e.g., isolate NRRL B-50349 from Bayer CropScience), Bacillus amyloliquefaciens TrigoCor (also known as “TrigoCor 1448”; e.g., isolate Embrapa Trigo Accession No. 144/88.4Lev, Cornell Accession No. Pma007BR-97, and ATCC accession No. 202152, from Cornell University, USA) and combinations thereof.

In still an even more particular embodiment, the one or more bacterial strains is a strain of Bacillus amyloliquefaciens. In an even more particular embodiment, the bacterial strain is Bacillus amyloliquefaciens strain PTA-7543 (previously classified as Bacillus atrophaeus), and/or Bacillus amyloliquefaciens strain NRRL B-50154. In one embodiment, the bacterial strain is Bacillus amyloliquefaciens strain PTA-7543 (previously classified as Bacillus atrophaeus). In another embodiment the bacterial strain is Bacillus amyloliquefaciens strain NRRL B-50154.

The fermentation of the one or more bacterial strains may be conducted using conventional fermentation processes, such as, aerobic liquid-culture techniques, shake flask cultivation, and small-scale or large-scale fermentation (e.g., continuous, batch, fed-batch, solid state fermentation, etc.) in laboratory or industrial fermentors, and such processes are well known in the art. Notwithstanding the production process used to produce the one or more bacterial strains, the one or more bacterial strains may be used directly from the culture medium or subject to purification and/or further processing steps (e.g., a drying process).

Following fermentation, the one or more bacterial strains may be recovered using conventional techniques (e.g., by filtration, centrifugation, etc.). The one or more bacterial strains may alternatively be dried (e.g., air-drying, freeze drying, or spray drying to a low moisture level, and storing at a suitable temperature, e.g., room temperature).

### Carriers

The carriers described herein will allow the microorganism(s) to remain efficacious (e.g., capable of enhancing plant growth, capable of expressing fungicidal activity, etc) and viable once formulated. Non-limiting examples of carriers described herein include liquids, slurries, or solids (including wettable powders or dry powders). In an embodiment, the carrier is a soil compatible carrier as described herein.

In one embodiment, the carrier is a liquid carrier. Non-limiting examples of liquids useful as carriers for the compositions disclosed herein include water, an aqueous solution, or a non-aqueous solution. In one embodiment, the carrier is water. In another embodiment the carrier is an aqueous solution, such as sugar water. In another embodiment, the carrier is a non-aqueous solution. If a liquid carrier is used, the liquid (e.g., water) carrier may further comprise growth media to culture the microorganisms described herein. Non-limiting examples of suitable growth media for the microorganisms described herein include arabinose-gluconate (AG), yeast extract mannitol (YEM), G16 media, or any media known to those skilled in the art to be compatible with, and/or provide growth nutrients to the strains.

In another embodiment, the carrier is a slurry. In an embodiment, the slurry may comprise a sticking agent, a liquid, or a combination thereof. It is envisioned that the sticking agent can be any agent capable of sticking the inoculum (e.g., one or more of the deposited strains) to a substrate of interest (e.g., a seed). Non-limiting examples of sticking agents include alginate, mineral oil, syrup, gum arabic, honey, methyl cellulose, milk, wallpaper paste, and combinations thereof. Non-limiting examples of liquids appropriate for a slurry include water or sugar water.

In another embodiment, the carrier is a solid. In a particular embodiment the solid is a powder. In one embodiment the powder is a wettable powder. In another embodiment, the powder is a dry powder. In another embodiment, the solid is a granule. Non-limiting examples of solids useful as carriers for the compositions disclosed herein include peat, wheat, wheat chaff, ground wheat straw, bran, vermiculite, cellulose, starch, soil (pasteurized or unpasteurized), gypsum, talc, clays (e.g., kaolin, bentonite, montmorillonite), and silica gels.

### Germinants

The compositions described herein may comprise one or more germinants. The one or more germinants described herein may be in either a liquid or solid form (including wettable powders or dry powders). In one embodiment, the germinant is in a liquid form. In another embodiment, the germinant is in a solid form. In a particular embodiment the germinant is a solid in the form of a powder. In another embodiment the powder is a wettable powder. In still another embodiment, the powder is a dry powder. In some examples, the germinants in a composition may be optional.

Non-limiting examples of germinants that may be suitable for the compositions described herein include lactate; lactose (as found in dairy products), bicarbonate or carbonate compounds such as sodium bicarbonate; carbon dioxide (e.g., carbonic acid: CO₂ dissolved in water, as is common in “sodas” or “soft drinks” such as cola or some fruit flavored beverages); compounds that adsorb lipid (e.g., starch, such as found in wheat, rice or other grains and potatoes and some other vegetables); charcoal or similar materials of high surface area that may adsorb or absorb fatty acid and lipid materials that may inhibit spore germination; monosaccharides such as fructose, glucose, mannose, or galactose; alanine, asparagine, cysteine, glutamine, norvatine, serine, threonine, valine, glycine, or other amino acid, and derivatives thereof such as N-(L-a-aspartyl)-L-phenylalanine (commonly sold under the trade name of “Aspartame”); inosine; bile salts such as taurocholate; and combinations of such spore germinants. For example, useful spore germinants can include alanine alone or in combination with lactate; a combination of L-asparagine, glucose, fructose, and potassium ion (AGFK); amino acids such as aspargine, cysteine, or serine alone or in combination with lactate; and caramels created by autoclaving monosaccharides or such caramels in combination with amino acids. In one embodiment, the composition comprises one or more germinants. In a particular embodiment, the composition comprises L-asparagine, glucose, fructose, and potassium ion (AGFK).

In a particular embodiment, the one or more germinants will be present in a concentration of 0.001 mM to 10.0 M of the composition, particularly 0.01 mM to 5.0 M of the composition, and more particularly 0.1 mM to 1.0 M of the composition. In a more particular embodiment the one or more germinants will be present in a concentration between 1.0 mM to 0.1 M of the composition.

### Animal Feed

The treated bacterial spores of the invention are suitable for use in animal feed(s), and may be added to animal feed compositions, as described in for example WO 2014/169046.

The characteristics of the compositions described herein allow its use as a component which is well suited for inclusion with an animal feed. In particular embodiments, the compositions described herein are mixed with an animal feed ingredient and/or animal feed(s) and referred to as a mash feed. In certain embodiments, the mash feed is subsequently pelletized.

The animal feed may comprise any ingredient suitable for intake by aquatic animals, e.g., comprising sources of protein, lipids, carbohydrates, salts, minerals and vitamins. The animal feed ingredients may be selected, and mixed in any proportions, suitable to meet the nutritional needs of the aquatic animals to be fed with the feed and/or to keep the raw material cost of the feed within desired limits and/or to achieve other desired properties of the feed. Non-limiting examples of animal feed ingredients may include one or more of the following materials: plant derived products, such as seeds, grains, leaves, roots, tubers, flowers, pods, husks, oil, soybean meal, soy protein isolate, potato protein powder, wheat, barley, corn, soybean oil, and corn gluten meal; animal derived products, such as fish meal, fish oil, milk powder, skim milk powder, bone extract, meat extract, blood extract, and the like; additives, such as minerals, vitamins, aroma compounds, and feed enhancing enzymes.

In particular embodiments, the animal feed may comprise 0-80% maize; and/or 0-80% sorghum; and/or 0-70% wheat; and/or 0-70% barley; and/or 0-30% oats; and/or 0-40% soybean meal; and/or 0-10% fish meal; and/or 0-20% whey.

The animal feed may comprise vegetable proteins. In particular embodiments, the protein content of the vegetable proteins is at least 10, 20, 30, 40, 50, 60, 70, 80, or 90% (w/w). Vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, and combinations thereof.

In a particular embodiment, the vegetable protein source is material from one or more plants of the family Fabaceae, e.g., soybean, lupine, pea, or bean. In another particular embodiment, the vegetable protein source is material from one or more plants of the family Chenopodiaceae, e.g. beet, sugar beet, spinach or quinoa. Other examples of vegetable protein sources are rapeseed, and cabbage. In another particular embodiment, soybean is a preferred vegetable protein source. Other examples of vegetable protein sources are cereals such as barley, wheat, rye, oat, maize (corn), rice, and sorghum.

In another embodiment, the animal feed may optionally comprise one or more suitable animal feed additives. Non-limiting examples of suitable animal feed additives include enzyme inhibitors, fat-soluble vitamins, water soluble vitamins, trace minerals, macro minerals, and combinations thereof.

In another embodiment, the animal feed may further optionally comprise one or more feed-additive ingredients. Non-limiting examples of feed-additive ingredients include colouring agents, aroma compounds, stabilisers, anti-microbial peptides (non-limiting examples of anti-microbial peptides (AMP's) are CAP18, Leucocin A, Tritrpticin, Protegrin-1, Thanatin, Defensin, Ovispirin such as Novispirin (Robert Lehrer, 2000), and variants, or fragments thereof which retain antimicrobial activity), anti-fungal polypeptides (AFP's) (non-limiting examples include the Aspergillus giganteus, and Aspergillus niger peptides, as well as variants and fragments thereof which retain antifungal activity, as disclosed in WO 94/01459 and PCT/DK02/00289), and/or at least one other enzyme selected from amongst phytases EC 3.1.3.8 or 3.1.3.26; xylanases EC 3.2.1.8; galactanases EC 3.2.1.89; and/or beta-glucanases EC 3.2.1.4.

In still another embodiment, the animal feed may still further optionally include one or more fat- and water soluble vitamins, trace minerals and macro minerals. Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D3, vitamin E, and vitamin K, e.g., vitamin K3.

Non-limiting examples of water-soluble vitamins include vitamin B12, biotin and choline, vitamin B1, vitamin B2, vitamin B6, niacin, folic acid and panthothenate, e.g., Ca-D-panthothenate.

Non-limiting examples of trace minerals include boron, cobalt, chloride, chromium, copper, fluoride, iodine, iron, manganese, molybdenum, selenium, zinc, etc.

Non-limiting examples of macro minerals include calcium, magnesium, potassium, sodium, etc.

### Agricultural Compositions

The treated bacterial spores of the invention may be added to and thus become a component of an agricultural composition, and be used in an agricultural application, as described in for example WO 2014/193746.

In addition to the treated bacterial spores, the agricultural compositions comprise a carrier and optionally one or more germinants. The composition may be in the form of a liquid, a gel, a slurry, a solid, or a powder (wettable powder or dry powder). In a particular embodiment, the composition is a dry or substantially dry composition. As used herein, the term “substantially dry composition(s)” is understood to be a composition containing less than 20 wt. % of free water, more preferably less than 10 wt. % of free water, even more preferably less than 5 wt. % of free water, still even more preferably less than 2.5 wt. % of free water, most preferably less than 1 wt. % of free water.

Dry compositions, as described herein, may be suitable for mixing with one or more liquids for formulation of a liquid product for foliar application to a plant or plant part, a seed treatment, an in furrow treatment, or a combination thereof. In yet another embodiment, the dry composition comprises microorganisms that remain in a spore form in the presence of a germinant until the dry composition is formulated (e.g., the composition is mixed and/or combined) with one or more solvents. Solvents may be aqueous or organic. Representative examples of solvents that may be suitable for use in certain embodiments include water or an organic solvent such as isopropyl alcohol or a glycol ether.

The carriers described herein will allow the microorganism(s) to remain efficacious (e.g., capable of enhancing plant growth, capable of expressing fungicidal activity, etc) and viable once formulated. Non-limiting examples of carriers described herein include liquids, slurries, or solids (including wettable powders or dry powders). In an embodiment, the carrier is a soil compatible carrier as described herein.

In one embodiment, the carrier is a liquid carrier. Non-limiting examples of liquids useful as carriers for the compositions disclosed herein include water, an aqueous solution, or a non-aqueous solution. In one embodiment, the carrier is water. In another embodiment the carrier is an aqueous solution, such as sugar water. In another embodiment, the carrier is a non-aqueous solution. If a liquid carrier is used, the liquid (e.g., water) carrier may further comprise growth media to culture the microorganisms described herein. Non-limiting examples of suitable growth media for the microorganisms described herein include arabinose-gluconate (AG), yeast extract mannitol (YEM), G16 media, or any media known to those skilled in the art to be compatible with, and/or provide growth nutrients to the strains.

In another embodiment, the carrier is a slurry. In an embodiment, the slurry may comprise a sticking agent, a liquid, or a combination thereof. It is envisioned that the sticking agent can be any agent capable of sticking the inoculum (e.g., one or more of the deposited strains) to a substrate of interest (e.g., a seed). Non-limiting examples of sticking agents include alginate, mineral oil, syrup, gum arabic, honey, methyl cellulose, milk, wallpaper paste, and combinations thereof. Non-limiting examples of liquids appropriate for a slurry include water or sugar water.

In another embodiment, the carrier is a solid. In a particular embodiment the solid is a powder. In one embodiment the powder is a wettable powder. In another embodiment, the powder is a dry powder. In another embodiment, the solid is a granule. Non-limiting examples of solids useful as carriers for the compositions disclosed herein include peat, wheat, wheat chaff, ground wheat straw, bran, vermiculite, cellulose, starch, soil (pasteurized or unpasteurized), gypsum, talc, clays (e.g., kaolin, bentonite, montmorillonite), and silica gels.

The compositions disclosed herein may comprise one or more agriculturally beneficial ingredients. Alternatively, as persons skilled in the art would appreciate, any one or more of these agents may be used in the methods described herein via separate composition or formulation. Non-limiting examples of agriculturally beneficial ingredients include one or more biologically active ingredients, nutrients, biostimulants, preservatives, polymers, wetting agents, surfactants, herbicides, fungicides, insecticides, or combinations thereof.

Methods of using the agricultural compositions include treating a plant or plant part comprising contacting a plant or plant part with the (one or more) treated bacterial spores of the invention and one or more germinants. In one embodiment, the plant or plant part is contacted by the one or more bacterial spores sequentially (i.e., before or after) with the one or more germinants. In another embodiment, the plant or plant part is contacted by the one or more bacterial spores simultaneously (i.e., at or about the same time) with the one or more germinants. In a particular embodiment the method includes treating a plant or plant part comprising contacting a plant or plant part with one or more compositions described herein.

The applying step can be performed by any method known in the art (including both foliar and non-foliar applications). Non-limiting examples of applying to the plant or plant part include spraying a plant or plant part, drenching a plant or plant part, dripping on a plant or plant part, dusting a plant or plant part, and/or coating a seed. In a more particular embodiment, the applying step is repeated (e.g., more than once, as in the contacting step is repeated twice, three times, four times, five times, six times, seven times, eight times, nine times, ten times, etc.).

In a particular embodiment the contacting step comprises foliarly applying to a plant or plant part (i.e., application to the plant by spraying, e.g., via foliar spray, a predosage device, a knapsack sprayer, a spray tank or a spray plane) one or more bacterial spores and one or more germinants. In still yet a more particular embodiment, the contacting step comprises foliarly applying one or more compositions described herein to plant foliage.

In another embodiment, the method further comprises applying to the plant or plant part one or more agriculturally beneficial ingredients described herein. In one embodiment the one or more agriculturally beneficial ingredients are applied simultaneously or sequentially with the one or more bacterial spores. In another embodiment the one or more agriculturally beneficial ingredients are applied simultaneously or sequentially with the one or more germinants.

Application of the one or more agriculturally beneficial ingredients may also be applied to the plant or plant parts as part of a composition described herein or applied independently from the one or more compositions described herein. In one embodiment, the one or more agriculturally beneficial ingredients are applied to the plant or plant parts as part of one or more of the compositions described herein. In another embodiment, the one or more agriculturally beneficial ingredients are applied to the plant or plant parts independently from the one or more compositions described herein. In one embodiment, the step of applying the one or more agriculturally beneficial ingredients to the plant or plant part occurs before, during, after, or simultaneously with the step of contacting a plant or plant part with one or more of the compositions described herein.

In a yet another aspect, a method for inducing the germination of a bacterial spore is described herein. In one embodiment, the method comprises inducing the germination of a microorganism comprising foliarly applying one or more bacterial spores and one or more germinants to a plant or plant part, wherein upon foliar application of the one or more bacterial spores and the one or more germinants to a plant or plant part, the one or more bacterial spores exhibit increased germination on the plant or plant part in the presence of the one or more germinants compared to the foliar application of one or more bacterial spores alone (i.e., without one or more germinants) on a plant or plant part. As used herein, the terms “increased germination” “enhanced germination” and/or variations thereof, is intended to mean an increase in the proportion of applied spores that germinate in the presence of a germinant when compared to the proportion of applied spores that germinate in the absence of a germinant; the increase in speed by which applied spores germinate in the presence of a germinant when compared to the speed by which applied spores germinate in the absence of a germinant, or combinations thereof. In a more particular embodiment, the method for inducing germination of a bacterial spore comprises foliarly applying one or more bacterial spores and one or more germinants to plant foliage. In still another embodiment, the method for inducing germination of a bacterial spore comprises foliarly applying one or more compositions described herein.

The method may further comprise subjecting the plant or plant part to one or more agriculturally beneficial ingredients, applied simultaneously or sequentially with the one or more bacterial spores or one or more germinants. In one embodiment the one or more agriculturally beneficial ingredients are applied simultaneously or sequentially with the one or more bacterial spores. In another embodiment the one or more agriculturally beneficial ingredients are applied simultaneously or sequentially with the one or more germinants. Application of the one or more agriculturally beneficial ingredients may also be applied to the plant or plant parts as part of a composition described herein or applied independently from the one or more compositions described herein. In one embodiment, the one or more agriculturally beneficial ingredients are applied to the plant or plant parts as part of one or more of the compositions described herein. In another embodiment, the one or more agriculturally beneficial ingredients are applied to the plant or plant parts independently from the one or more compositions described herein. In one embodiment, the step of applying the one or more agriculturally beneficial ingredients to the plant or plant part occurs before, during, after, or simultaneously with the step of contacting a plant or plant part with one or more of the compositions described herein.

In another aspect, a method for treating soil is described herein. In one embodiment, the method comprises contacting a soil with one or more bacterial spores and one or more germinants. In another embodiment, the method comprises contacting a soil with one or more bacterial spores and one or more germinants, and growing a plant or plant part in the treated soil. In still yet another embodiment, the method comprises contacting a soil with one or more of the compositions described herein, and growing a plant or plant part in the treated soil.

In an embodiment, the contacting step can be performed by any method known in the art. Non-limiting examples of contacting the soil include spraying the soil, drenching the soil, dripping onto the soil, and/or dusting the soil. In one embodiment, the contacting step is repeated (e.g., more than once, as in the contacting step is repeated twice, three times, four times, five times, six times, seven times, eight times, nine times, ten times, etc.). In one embodiment, the contacting step comprises contacting the soil with one or more bacterial spores sequentially with one or more germinants. In another embodiment, the contacting step comprises contacting the soil with one or more bacterial spores simultaneously with one or more germinants. In a particular embodiment, the contacting step comprises introducing one or more of the compositions described herein to the soil.

The contacting step can occur at any time during the growth of the plant or plant part. In one embodiment, the contacting step occurs before the plant or plant part begins to grow. In another embodiment, the contacting step occurs after the plant or plant part has started to grow.

In another embodiment, the method further comprises the step of planting a plant or plant part. The planting step can occur before, after or during the contacting step. In one embodiment, the planting step occurs before the contacting step. In another embodiment, the planting step occurs during the contacting step (e.g., the planting step occurs simultaneously with the contacting step, the planting step occurs substantially simultaneous with the contacting step, etc.). In still another embodiment, the planting step occurs after the contacting step.

The method may further comprise subjecting the soil to one or more agriculturally beneficial ingredients, applied simultaneously or sequentially with the one or more bacterial spores or one or more germinants. In one embodiment the one or more agriculturally beneficial ingredients are applied simultaneously or sequentially with the one or more bacterial spores. In another embodiment the one or more agriculturally beneficial ingredients are applied simultaneously or sequentially with the one or more germinants. Application of the one or more agriculturally beneficial ingredients may also be applied to the soil as part of a composition described herein or applied independently from the one or more compositions described herein. In one embodiment, the one or more agriculturally beneficial ingredients are applied to the soil as of one or more of the compositions described herein. In another embodiment, the one or more agriculturally beneficial ingredients are applied to the soil independently from the one or more compositions described herein. In one embodiment, the step of applying the one or more agriculturally beneficial ingredients to the plant or plant part occurs before, during, after, or simultaneously with the step of contacting a plant or plant part with one or more of the compositions described herein.

In one embodiment, the step of subjecting the soil to one or more agriculturally beneficial ingredients occurs sequentially or simultaneously with the contacting step. In one embodiment, the step of subjecting the soil to one or more agriculturally beneficial ingredients as described herein occurs before the contacting step. In another embodiment, the step of subjecting the soil to one or more agriculturally beneficial ingredients as described herein occurs during the contacting step. In still another embodiment, the step of subjecting the soil to one or more agriculturally beneficial ingredients as described herein occurs after the contacting step. In yet another embodiment, the step of subjecting the soil to one or more agriculturally beneficial ingredients as described herein occurs simultaneously with the contacting step (e.g., contacting the soil with one or more of the compositions described herein, etc.).

The methods described herein are applicable to both leguminous and non-leguminous plants or plant parts. In a particular embodiment the plants or plant parts are selected from the group consisting of alfalfa, rice, wheat, barley, rye, oat, cotton, canola, sunflower, peanut, corn, potato, sweet potato, bean, pea, chickpeas, lentil, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane.

### Detergent Compositions

The treated bacterial spores of the invention may be added to and thus become a component of a detergent or cleaning composition, such as described in for example WO 2012/112718. Thus, a composition for inhibiting malodor in a cleaning machine, cleaning process or article treated (cleaned) in a cleaning machine or cleaning process is also provided.

The detergent composition of the invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

In a specific aspect, the invention provides a detergent additive comprising the treated bacterial spores of the invention, as described herein.

In one embodiment, the invention is directed to detergent compositions comprising the treated bacterial spores of the present invention in combination with one or more additional cleaning composition components. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

In one embodiment of the present invention, the treated bacterial spores of the present invention may be added to a detergent composition in an amount corresponding to 0.001-200 mg of enzyme protein, such as 0.005-100 mg of enzyme protein, preferably 0.01-50 mg of enzyme protein, more preferably 0.05-20 mg of enzyme protein, even more preferably 0.1-10 mg of enzyme protein per liter of wash liquor.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized.

When included therein the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the detergent will usually contain from about 0.1% to about 10% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein the detergent will usually contain from about 0.1% to about 20% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein the detergent will usually contain from about 0.1% to about 10% by weight of a zwitterionic surfactant. Non-limiting examples of zwitterionic surfactants include betaine, alkyldimethylbetaine, sulfobetaine, and combinations thereof.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however, the molecular structure of hydrotropes generally do not favor spontaneous self-aggregation, see for example review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with calcium and magnesium ions. Any builder and/or co-builder known in the art for use in laundry detergents may be utilized. Non-limiting examples of builders include citrates, zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also contain 0-50% by weight, such as about 5% to about 30%, of a detergent co-builder, or a mixture thereof. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N, N′, N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 2009/102854, U.S. Pat. No. 5,977,053.

### Bleaching Systems

The detergent may contain 0-50% by weight of a bleaching system. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO 1998/017767. A particular family of bleach activators of interest was disclosed in EP624154 and particularly preferred in that family is acetyl triethyl citrate (ATC). ATC or a short chain triglyceride like triacetin has the advantage that it is environmental friendly as it eventually degrades into citric acid and alcohol. Furthermore acetyl triethyl citrate and triacetin has a good hydrolytical stability in the product upon storage and it is an efficient bleach activator. Finally ATC provides a good building capacity to the laundry additive. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

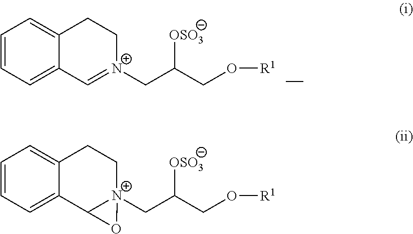

and mixtures thereof; wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl. Other exemplary bleaching systems are described, e.g., in WO 2007/087258, WO 2007/087244, WO 2007/087259 and WO 2007/087242. Suitable photobleaches may for example be sulfonated zinc phthalocyanine.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575 and U.S. Pat. No. 5,955,415. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/03274, WO 2005/03275, WO 2005/03276 and EP 1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g., WO 2007/087257 and WO 2007/087243.

### Detergent Enzyme(s)

The detergent additive as well as the detergent composition may comprise one or more enzymes suitable for including in laundry or dishwash detergents (detergent enzymes), such as a protease (e.g., subtilisin or metalloprotease), lipase, cutinase, amylase, carbohydrase, cellulase, pectinase, mannanase, arabinase, galactanase, xanthanase (EC 4.2.2.12), xylanase, DNAse, perhydrolase, oxidoreductase (e.g., laccase, peroxidase, peroxygenase and/or haloperoxidase). Preferred detergent enzymes are protease (e.g., subtilisin or metalloprotease), lipase, amylase, lyase, cellulase, pectinase, mannanase, DNAse, perhydrolase, and oxidoreductases (e.g., laccase, peroxidase, peroxygenase and/or haloperoxidase); or combinations thereof. More preferred detergent enzymes are protease (e.g., subtilisin or metalloprotease), lipase, amylase, cellulase, pectinase, and mannanase; or combinations thereof.

Proteases:

The proteases for use in the present invention are serine proteases, such as subtilisins, metalloproteases and/or trypsin-like proteases. Preferably, the proteases are subtilisins or metalloproteases; more preferably, the proteases are subtilisins.

A serine protease is an enzyme which catalyzes the hydrolysis of peptide bonds, and in which there is an essential serine residue at the active site (White, Handler and Smith, 1973 “Principles of Biochemistry,” Fifth Edition, McGraw-Hill Book Company, NY, pp. 271-272). Subtilisins include, preferably consist of, the I-S1 and I-S2 sub-groups as defined by Siezen et al., Protein Engng. 4 (1991) 719-737; and Siezen et al., Protein Science 6 (1997) 501-523. Because of the highly conserved structure of the active site of serine proteases, the subtilisin according to the invention may be functionally equivalent to the proposed sub-group designated subtilase by Siezen et al. (supra).

The subtilisin may be of animal, vegetable or microbial origin, including chemically or genetically modified mutants (protein engineered variants), preferably an alkaline microbial subtilisin. Examples of subtilisins are those derived from Bacillus, e.g., subtilisin Novo, subtilisin Carlsberg, subtilisin BPN′, subtilisin 309, subtilisin 147 and subtilisin 168 (described in WO 1989/06279) and Protease PD138 (WO 1993/18140). Examples are described in WO 1998/020115, WO 2001/44452, WO 2001/58275, WO 2001/58276, WO 2003/006602 and WO 2004/099401. Examples of trypsin-like proteases are trypsin (e.g., of porcine or bovine origin) and the Fusarium protease described in WO 1989/06270 and WO 1994/25583. Other examples are the variants described in WO 1992/19729, WO 1988/08028, WO 1998/20115, WO 1998/20116, WO 1998/34946, WO 2000/037599, WO 2011/036263, especially the variants with substitutions in one or more of the following positions: 27, 36, 57, 76, 87, 97, 101, 104, 120, 123, 167, 170, 194, 206, 218, 222, 224, 235, and 274.

The metalloprotease may be of animal, vegetable or microbial origin, including chemically or genetically modified mutants (protein engineered variants), preferably an alkaline microbial metalloprotease. Examples are described in WO 2007/044993, WO 2012/110562 and WO 2008/134343.

Examples of commercially available subtilisins include Kannase™, Everlase™, Relase™, Esperase™, Alcalase™, Durazym™, Savinase™, Ovozyme™, Liquanase™, Coronase™, Polarzyme™, Pyrase™, Pancreatic Trypsin NOVO (PTN), Bio-Feed™ Pro and Clear-Lens™ Pro; Blaze (all available from Novozymes A/S, Bagsvaerd, Denmark). Other commercially available proteases include Neutrase™, Ronozyme™ Pro, Maxatase™, Maxacal™, Maxapem™, Opticlean™, Properase™, Purafast™, Purafect™, Purafect Ox™, Purafact Prime™, Excellase™, FN2™, FN3™ and FN4™ (available from Novozymes, Genencor International Inc., Gist-Brocades, BASF, or DSM). Other examples are Primase™ and Duralase™. Blap R, Blap S and Blap X available from Henkel are also examples.

Lyases:

The lyase may be a pectate lyase derived from Bacillus, particularly B. lichemiformis or B. agaradhaerens, or a variant derived of any of these, e.g. as described in U.S. Pat. No. 6,124,127, WO 1999/027083, WO 1999/027084, WO 2002/006442, WO 2002/092741, WO 2003/095638, Commercially available pectate lyases are XPect; Pectawash and Pectaway (Novozymes A/S).

Mannanase:

The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 99/064619. A commercially available mannanase is Mannaway (Novozymes A/S).

Cellulases:

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 1989/09259.

Especially suitable cellulases are the alkaline or neutral cellulases having color care benefits. Examples of such cellulases are cellulases described in EP 0 495 257, EP 0 531 372, WO 1996/011262, WO 1996/029397, WO 1998/008940. Other examples are cellulase variants such as those described in WO 1994/007998, EP 0 531 315, U.S. Pat. No. 5,457,046, U.S. Pat. No. 5,686,593, U.S. Pat. No. 5,763,254, WO 1995/024471, WO 1998/012307 and PCT/DK98/00299.

Commercially available cellulases include Celluzyme™, and Carezyme™ (Novozymes A/S), Clazinase™, and Puradax HA™ (Genencor International Inc.), and KAC-500(B)™ (Kao Corporation).

Lipases and Cutinases:

Suitable lipases and cutinases include those of bacterial or fungal origin. Chemically modified or protein engineered mutants are included. Examples include lipase from Thermomyces, e.g., from T. lanuginosus (previously named Humicola lanuginosa) as described in EP 258 068 and EP 305 216, cutinase from Humicola, e.g., H. insolens as described in WO 1996/013580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 1996/012012), a Bacillus lipase, e.g., from B. subtilis (Dartois et al., 1993, Biochemica et Biophysica Acta, 1131: 253-360), B. stearothermophilus (JP 64/744992) or B. pumilus (WO 1991/016422).

Other examples are lipase variants such as those described in WO 1992/005249, WO 1994/001541, EP 407 225, EP 260 105, WO 1995/035381, WO 1996/000292, WO 1995/030744, WO 1994/025578, WO 1995/014783, WO 1995/022615, WO 1997/004079, WO 1997/007202, WO 2000/060063, WO 2007/087508 and WO 2009/109500.

Preferred commercially available lipase enzymes include Lipolase™, Lipolase Ultra™, and Lipex™; Lecitase™, Lipolex™; Lipoclean™, Lipoprime™ (Novozymes A/S). Other commercially available lipases include Lumafast (Genencor Int Inc); Lipomax (Gist-Brocades/Genencor Int Inc) and Bacillus sp. lipase from Solvay.

Amylases:

Suitable amylases (α and/or β) include those of bacterial or fungal origin. Chemically modified or protein engineered mutants are included. Amylases include, for example, α-amylases obtained from Bacillus, e.g., a special strain of Bacillus licheniformis, described in more detail in GB 1,296,839.

Examples of suitable amylases include amylases having SEQ ID NO: 2 in WO 1995/010603 or variants having 90% sequence identity to SEQ ID NO: 3 thereof. Preferred variants are described in WO 1994/002597, WO 1994/018314, WO 1997/043424 and SEQ ID NO: 4 of WO 1999/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 2002/010355 or variants thereof having 90% sequence identity to SEQ ID NO: 6. Preferred variants of SEQ ID NO: 6 are those having a deletion in positions 181 and 182 and a substitution in position 193. Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

### M197T;

### H156Y+A181T+N190F+A209V+Q264S; or

### G48A+T49I+G107A+H156Y+A181T+N190F+I201F+A209V+Q264S.

Further amylases which are suitable are amylases having SEQ ID NO: 6 in WO 1999/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 1996/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 2008/153815, SEQ ID NO: 10 in WO 2001/66712 or variants thereof having 90% sequence identity to SEQ ID NO: 2 of WO 2008/153815 or 90% sequence identity to SEQ ID NO: 10 in WO 2001/066712. Preferred variants of SEQ ID NO: 10 in WO 2001/066712 are those having a substitution, a deletion or an insertion in one of more of the following positions: 176, 177, 178, 179, 190, 201, 207, 211 and 264.

Further suitable amylases are amylases having SEQ ID NO: 2 of WO 2009/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T1311, T1651, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

### N128C+K178L+T182G+Y305R+G475K;

### N128C+K178L+T182G+F202Y+Y305R+D319T+G475K;

### S125A+N128C+K178L+T182G+Y305R+G475K; or

S125A+N128C+T1311+T1651+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in WO 2001/066712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO 2011/098531, WO 2013/001078 and WO 2013/001087.

Commercially available amylases are Stainzyme; Stainzyme Plus; Duramyl™, Termamyl™, Termamyl Ultra; Natalase, Fungamyl™ and BAN™ (Novozymes A/S), Rapidase™ and Purastar™/Effectenz™, Powerase and Preferenz S100 (from Genencor International Inc./DuPont).

Deoxyribonuclease (DNase):

Suitable deoxyribonucleases (DNases) are any enzyme that catalyzes the hydrolytic cleavage of phosphodiester linkages in the DNA backbone, thus degrading DNA. According to the invention, a DNase which is obtainable from a bacterium is preferred; in particular, a DNase which is obtainable from a Bacillus is preferred; in particular, a DNase which is obtainable from Bacillus subtilis or Bacillus licheniformis is preferred. Examples of such DNases are described in patent application WO 2011/098579 or in PCT/EP2013/075922.

Perhydrolases:

Suitable perhydrolases are capable of catalyzing a perhydrolysis reaction that results in the production of a peracid from a carboxylic acid ester (acyl) substrate in the presence of a source of peroxygen (e.g., hydrogen peroxide). While many enzymes perform this reaction at low levels, perhydrolases exhibit a high perhydrolysis:hydrolysis ratio, often greater than 1. Suitable perhydrolases may be of plant, bacterial or fungal origin. Chemically modified or protein engineered mutants are included.

Examples of useful perhydrolases include naturally occurring Mycobacterium perhydrolase enzymes, or variants thereof. An exemplary enzyme is derived from Mycobacterium smegmatis. Such enzyme, its enzymatic properties, its structure, and variants thereof, are described in WO 2005/056782, WO 2008/063400, US 2008/145353, and US 2007/167344.

Oxidases/Peroxidases:

Suitable oxidases and peroxidases (or oxidoreductases) include various sugar oxidases, laccases, peroxidases and haloperoxidases.

Suitable peroxidases include those comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 1993/024618, WO 1995/010602, and WO 1998/015257.

A peroxidase for use in the invention also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 1995/027046; or C. verruculosa CBS 147.63 or C. verruculosa CBS 444.70 as described in WO 1997/004102; or from Drechslera hartlebii as described in WO 2001/079459, Dendryphiella salina as described in WO 2001/079458, Phaeotrichoconis crotalarie as described in WO 2001/079461, or Geniculosporium sp. as described in WO 2001/079460.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 19920/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 1997/008325; or from Myceliophthora thermophila, as disclosed in WO 1995/033836.

Examples of other oxidases include, but are not limited to, amino acid oxidase, glucose oxidase, lactate oxidase, galactose oxidase, polyol oxidase (e.g., WO 2008/051491), and aldose oxidase. Oxidases and their corresponding substrates may be used as hydrogen peroxide generating enzyme systems, and thus a source of hydrogen peroxide. Several enzymes, such as peroxidases, haloperoxidases and perhydrolases, require a source of hydrogen peroxide. By studying EC 1.1.3._, EC 1.2.3._, EC 1.4.3._, and EC 1.5.3._{—} or similar classes (under the International Union of Biochemistry), other examples of such combinations of oxidases and substrates are easily recognized by one skilled in the art.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

The detergent enzyme(s) may be included in a detergent composition by adding separate additives containing one or more enzymes, or by adding a combined additive comprising all of these enzymes.

### Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants—

The detergent compositions of the present invention can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents—

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent Whitening Agent—

The detergent compositions of the present invention will preferably also contain additional components that may tint articles being cleaned, such as fluorescent whitening agent or optical brighteners. Fluorescent whitening agents, also referred to as optical brighteners, optical brightening agents, or fluorescent brightening agents, are dyes that absorb light in the ultraviolet and violet region (usually 340-370 nm) of the electromagnetic spectrum, and re-emit light in the blue region (typically 420-470 nm). These agents are often used to enhance the appearance of color of fabric and paper, causing a whitening effect, making materials look less yellow by increasing the overall amount of blue light reflected.

Fluorescent whitening agents are well known in the art, and many such fluorescent agents are available commercially. Usually, fluorescent agents are supplied and used in the form of their alkali metal salts, for example, the sodium salts.

Preferred fluorescent agents are selected from the classes, distyrylbiphenyls, triazinylaminostilbenes, bis(1,2,3-triazol-2-yl)stilbenes, bis(benzo[b]furan-2-yl)biphenyls, 1,3-diphenyl-2-pyrazolines, thiophenediyl benzoxazole, and courmarins. The fluorescent agent is preferably sulfonated.

Preferred classes of fluorescent agent are: di-styryl biphenyl compounds, e.g., Tinopal™ CBS-X; di-amine stilbene di-sulphonic acid compounds, e.g., Tinopal DMS-X and Blankophor™ HRH; pyrazoline compounds, e.g., Blankophor SN; and thiophenediyl benzoxazole compounds, e.g., Tinopal OB. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2,2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS and Tinopal OB, available from BASF. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Tinopal OB is 2,5-thiophenediylbis(5-tert-butyl-1,3-benzoxazole). Another preferred fluorescent whitening agent is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent agents for use in the invention are also described in McElhone, H. J. (2009), “Fluorescent Whitening Agents”, Kirk-Othmer Encyclopedia of Chemical Technology, 1-16, DOI: 10.1002/0471238961.0612211513030512.a01.pub2.

Suitable fluorescent brightener levels include lower levels of from about 0.01, from 0.05, from about 0.1 or even from about 0.2 wt % to upper levels of 0.5 or even 0.75 wt %; such as from 0.01 wt % to 0.5 wt %.

Soil Release Polymers—

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalate based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-Redeposition Agents—

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other Suitable Adjunct Materials include, but are not limited to, anti-shrink agents, anti-wrinkling agents, bactericides, binders, carriers, dyes, enzyme stabilizers, fabric softeners, fillers, foam regulators, perfumes, pigments, sod suppressors, solvents, and structurants for liquid detergents and/or structure elasticizing agents.

### Laundry Soap Bars

The treated bacterial spores of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e., if a solid object (e.g., laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants, e.g., anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g., a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, a treated bacterial spore of the invention, optionally one or more enzymes, a protease inhibitor, and a salt of a monovalent cation and an organic anion may be prepared and the mixture is then plodded. The enzyme and optional additional enzymes may be added at the same time as the protease inhibitor for example in liquid form. Besides the mixing step and the plodding step, the process may further comprise the steps of milling, extruding, cutting, stamping, cooling and/or wrapping.

### Compositions, Methods and Uses

In a first aspect, the invention provides a stabilized bacterial spore composition comprising

(a) a carrier;
(b) optionally one or more germinants; and
(c) a bacterial spore population which has been treated with a sub-lethal heat treatment at 50-80° C. for more than 30 minutes followed by cooling to below 30° C.;
wherein the bacterial spore population exhibits improved germination after 24 hours compared to a non-treated, but otherwise identical, bacterial spore population.

In an embodiment, the composition is a substantially dry composition.

In an embodiment, the bacterial spore population exhibits improved germination after 7 days compared to a non-treated bacterial spore population.

In an embodiment, the heat treatment is carried out in an aqueous environment.

In an embodiment, the heat treatment is carried out at 60-75° C. for 30-240 minutes followed by cooling to room temperature.

In an embodiment, the bacterial spores are Bacillus spores.

In an embodiment, the composition is an animal feed composition and further comprises one or more animal feed additives. Preferably, the animal feed composition is an aquatic animal feed composition; more preferably, a shrimp feed composition or a salmon feed composition.

The aquatic animal feed composition may be used in a method for providing vegetative bacterial cells of a bacterial spore population in the gut of an aquatic animal, comprising feeding the aquatic animal with the aquatic animal feed composition as described above.

In another embodiment of the bacterial spore composition, the composition is a cleaning composition and further comprises a surfactant or a wetting agent, and/or a detergent builder.

The cleaning composition may be used in a method for inhibiting or preventing malodor in a laundry washing machine, comprising contacting the laundry washing machine with the cleaning composition as described above.

In another embodiment of the bacterial spore composition, the composition is an agricultural composition and further comprises one or more agriculturally beneficial ingredients.

The agricultural composition may be used for treating a plant or plant part comprising contacting the plant or plant part with the agricultural composition as described above.

In another aspect, the invention provides a method for preparing a stabilized bacterial spore composition comprising the steps of:

(a) treating a bacterial spore population with a sub-lethal heat treatment at 50-80° C. for more than 30 minutes followed by cooling to below 30° C.;
(b) mixing the treated bacterial spore population with a carrier, and optionally one or more germinants; and
(c) storing the bacterial spore population for at least 24 hours before or after step (b); wherein the bacterial spore population exhibits improved germination after 24 hours compared to a bacterial spore population which did not receive the treatment in (a).

In an embodiment, the bacterial spore population exhibits improved germination after 7 days compared to a bacterial spore population which did not receive the treatment in (a).

The embodiments of the bacterial spore compositions, as described above, also apply to the method for preparing a stabilized bacterial spore composition. Thus, the invention also provides methods for preparing animal feed compositions, cleaning compositions, and agricultural compositions.

The present invention is further described by the following examples which should not be construed as limiting the scope of the invention.

### EXAMPLES

Chemicals were commercial products of at least reagent grade. Unless otherwise indicated the percentages in the Examples are weight based.

### Example 1

### Heat Priming of Bacillus Spore Germination

In this work, spore germination was measured for 6 different strains of Bacillus with and without heat activation (also termed priming) immediately after treatment and after several days of cold storage post-treatment. Germination kinetics of the spores were measured by determining how fast the spores initiated rapid dipicolinic acid (DPA) release and the percentage of the population that ultimately committed to germination. In this manner we were able to determine the speed at which the population responded to germinants and the proportion of the community that could respond. It was discovered that in many cases the benefits of heat activation persisted for at least several weeks after priming.

### Methods

### Endospore Preparation

Six strains of Bacillus were investigated for this study: SB3086 Bacillus subtilis, SB3130 Bacillus subtilis, SB3615 Bacillus amyloliquefaciens, SB3189 Bacillus pumilis, SB3002 Bacillus pumilis, and SB3112 Bacillus megaterium. The endospore (spore) form of each organism was used in the study and was generated by Novozymes using a fermentation method which is a trade secret; it will be referred to as Fermentation A. In the case of SB3086 and SB3615 a second batch of spores was also prepared with a fermentation method which is a trade secret and distinct from Fermentation A; it will be referred to as Fermentation B. Each spore preparation was washed in sterile 4° C. water by centrifuging (10,000×g, 1 minute), aspirating the supernatant from the pellet, and re-suspending with water three consecutive times. All washed spore preparations were then set to a concentration of 0.5 A₆₀₀ₙₘ as measured by optical density (Synergy H4 Multi-Mode Reader, Bio-Tek) in sterile 4° C. water.

### Heat Activation

Washed spore preparations were split into three aliquots. One aliquot was placed in a boiling water bath for 2 hours (boiled). The second aliquot was placed into a 65° C. water bath for 30 minutes (primed). The third aliquot was stored at 4° C. (unprimed). After their corresponding incubations the boiled and primed aliquots were cooled at room temperature for 30 m and then kept at 4° C. The day of treatment was termed day 0.

### Germination Assay

All boiled, primed, and unprimed spore aliquots were assayed for germination in the same manner. Spore germination was determined using Terbium chloride (TbCl₃) to detect the presence of dipicolinic acid (DPA). DPA is a compound that is unique to endospores and comprises ˜10% of the mass of a spore. After germination of a spore is triggered, the entire stock of DPA is rapidly released into the environment. The amount of DPA released over time is commonly used to assay the speed and efficiency of spore germination via TbCl₃. TbCl₃ will interact with extracellular DPA to form Tb-DPA, which is a fluorescent molecule.

A 30 μl volume of a spore aliquot was added to a well in a 96-well flat-bottom microtiter plate that contained a germinant solution (7.1 mM L-asparagine, 7.1 mM dextrose, 7.1 mM d-fructose, 7.1 mM KCl, 73 μM TbCl₃). Immediately after mixing the spore aliquot with the germinant solution, the plate was placed into a plate reader (Synergy H4 Multi-Mode Reader, Bio-Tek) and the sample was measured for the evolution of a fluorescent Tb-DPA product over time. Tb-DPA was excited at 270 nm and emitted light at 545 nm. All aliquots were tested in triplicate. Baseline control readings of each sample were generated by omitting the germinants in the germinant solution (asparagine, dextrose, d-fructose, and KCL) and were performed in duplicate.

All aliquots were tested immediately after a sample was heat activated (day 0), and then re-tested at 2, 7, and 30 days post-treatment. All aliquots were incubated at 4° C. between assays and received no other treatments after the initial day 0 treatment.

### Data Analysis

The boiled aliquots released all the DPA of a spore aliquot into solution and thus indicated the consequence of 100% germination for that sample. Because that value was determined for each aliquot, all data was normalized by taking the fluorescence of an experimental aliquot (primed or unprimed) and expressing its value as a percentage of the boiled aliquot's fluorescence. Thus, the percentage of released DPA was inferred to represent the percent germination for that aliquot. The mean of triplicate replicates of each aliquot were reported at every time point and the error was determined as the standard deviation of the mean.

To directly compare samples, the values for T_{lag} and Gₘₐₓ were calculated for each (see FIG. 1A). T_{lag} is the amount of time after exposure to germinants that a population of spores begins to rapidly release DPA into the environment. T_{lag} indicates how rapidly a population of spores is responding to germinants. Gₘₐₓ is the maximum percentage of DPA that a sample releases during the experimental timeframe normalized by the total possible DPA that can be released by the sample when boiled. Gₘₐₓ indicates the maximum percentage of the spore population that is capable of responding to germinants. It is possible for a spore population to demonstrate both altered T_{lag} and Gₘₐₓ (FIG. 1B), or a change in only one of those measures while the other is constant (FIG. 10).

### Results

### SB3086

SB3086 produced via fermentation method A did not demonstrate a significant reduction in T_{lag} when heat primed compared to an unprimed control (Table 1). This was consistent over the entire time course of the experiment. SB3086 spores produced via fermentation method B had a noticeable effect. At 0, 1, and 30 days after treatment, the heat primed spores were not capable of creating a measurable rapid DPA release point at all. At days 2 and 7 post-treatment, the T_{lag} of primed spores was significantly longer than the control.

The Gₘₐₓ for SB3086 was significantly lower for heat primed spores compared to an untreated control (Table 2). This was consistent regardless of the fermentation method. This reduction in efficiency was significantly different for all time points except 30 days post-treatment of the fermentation method A batch.

TABLE 1
T_{lag} of SB3086 spores with and without heat priming.

| Fermen- | | | Days Since Priming | | | | |
|---|---|---|---|---|---|---|---|
| tation | | Treatment | 0 | 1 | 2 | 7 | 30 |
| A | Mean T_{lag} (m) | Unprimed | n/a | 74 | 56 | 68 | 179 |
| | | Primed | n/a | 96 | 43 | 72 | 166 |
| | Δ T_{lag} | | n/a | 31% | −24% | 6% | −7% |
| B | Mean T_{lag} (m) | Unprimed | 68 | 78 | 67 | 71 | 107 |
| | | Primed | n/a | n/a | 103 | 108 | n/a |
| | Δ T_{lag} | | n/a | n/a | 54% | 53% | n/a |

TABLE 2
Gₘₐₓ of SB3086 spores with and without heat priming.

| Fermen- | | | Days Since Priming | | | | |
|---|---|---|---|---|---|---|---|
| tation | | Treatment | 0 | 1 | 2 | 7 | 30 |
| A | Mean Gₘₐₓ (%) | Unprimed | 4 | 31 | 46 | 39 | 10 |
| | | Primed | 6 | 19 | 31 | 25 | 11 |
| | Δ Gₘₐₓ | | 42% | −39% | −32% | −35% | 10% |
| B | Mean Gₘₐₓ (%) | Unprimed | 22 | 18 | 27 | 22 | 13 |
| | | Primed | 8 | 8 | 11 | 11 | 7 |
| | Δ Gₘₐₓ | | −64% | −53% | −58% | −48% | −48% |

### SB3615

SB3615 produced via both fermentation methods A and B demonstrated reduced T_{lag} values when heat primed spores were compared to an untreated control (Table 3). The priming-induced T_{lag} reduction was significant in all assays. The magnitude of T_{lag} reduction did not stay constant over time primarily because the unprimed spores demonstrated a general increase in response over time. Regardless, the primed spores maintained at least a 22% improvement in the amount of time it takes to release DPA after being exposed to germinants.

When heat primed, the Gₘₐₓ for SB3615 was also increased compared to an untreated control (Table 4). This was independent of fermentation method. The increase was significant in every tested instance, except for the day 7 post-treatment test for fermentation method A spores. 3615 spores prepared with fermentation method B were highly affected by priming and increased germination efficiency by in excess of 60% at all time points.

TABLE 3
T_{lag} of SB3615 spores with and without heat priming.

| Fermen- | | | Days Since Priming | | | | |
|---|---|---|---|---|---|---|---|
| tation | | Treatment | 0 | 1 | 2 | 7 | 30 |
| A | Mean T_{lag} (m) | Unprimed | 71 | 55 | 51 | 43 | 43 |
| | | Primed | 27 | 27 | 27 | 27 | 34 |
| | Δ T_{lag} | | −62% | −51% | −47% | −37% | −22% |
| B | Mean T_{lag} (m) | Unprimed | n/a | 76 | 48 | 54 | 72 |
| | | Primed | 90 | 42 | 32 | 39 | 52 |
| | Δ T_{lag} | | n/a | −45% | −33% | −27% | −28% |

TABLE 4
Gₘₐₓ of SB3615 spores with and without heat priming.

| Fermen- | | | Days Since Priming | | | | |
|---|---|---|---|---|---|---|---|
| tation | | Treatment | 0 | 1 | 2 | 7 | 30 |
| A | Mean Gₘₐₓ (%) | Unprimed | 46 | 51 | 53 | 56 | 61 |
| | | Primed | 66 | 70 | 66 | 58 | 73 |
| | Δ Gₘₐₓ | | 43% | 36% | 24% | 5% | 20% |
| B | Mean Gₘₐₓ (%) | Unprimed | 4 | 24 | 30 | 29 | 30 |
| | | Primed | 16 | 49 | 56 | 49 | 56 |
| | Δ Gₘₐₓ | | 262% | 101% | 89% | 68% | 84% |

### SB3130

SB3130 demonstrated reduced T_{lag} values when heat primed compared to an untreated control (Table 5). The priming-induced T_{lag} reduction was significant except at 30 days post-treatment. Within 7 days post-treatment, the primed spores have maintained at least a 35% improvement in the amount of time it takes to release DPA after being exposed to germinants.

When heat primed, the Gₘₐₓ for SB3130 was increased compared to an untreated control (Table 6). The increase was significant for up to 2 days post-treatment.

TABLE 5
T_{lag} of SB3130 spores with and without heat priming.

| Fermen- | | | Days Since Priming | | | | |
|---|---|---|---|---|---|---|---|
| tation | | Treatment | 0 | 1 | 2 | 7 | 30 |
| A | Mean T_{lag} (m) | Unprimed | 27 | 62 | 46 | 68 | 47 |
| | | Primed | 18 | 20 | 19 | 36 | 31 |
| | Δ T_{lag} | | −35% | −67% | −58% | −47% | −34% |

TABLE 6
Gₘₐₓ of SB3130 spores with and without heat priming.

| Fermen- | | | Days Since Priming | | | | |
|---|---|---|---|---|---|---|---|
| tation | | Treatment | 0 | 1 | 2 | 7 | 30 |
| A | Mean Gₘₐₓ (%) | Unprimed | 46 | 19 | 22 | 20 | 27 |
| | | Primed | 57 | 31 | 40 | 25 | 30 |
| | Δ Gₘₐₓ | | 22% | 60% | 83% | 21% | 13% |

### SB3189

SB3189 demonstrated premature germination at some point after heat priming and consequently has no data for T_{lag} (Table 7). Even though a minority of the spores committed to germinate prematurely, the presence of their DPA in the sample supernatant masked the T_{lag} of spores that remained dormant until germinant exposure.

Regardless of premature germination, a steady increase in DPA release did occur after germinant exposure for these samples and a Gₘₐₓ could be calculated (Table 8). When heat primed, the Gₘₐₓ for SB3189 was increased compared to an untreated control and that increase was significant at all time points except on day 2 post-treatment.

TABLE 7
T_{lag} of SB3189 spores with and without heat priming.

| Fermen- | | | Days Since Priming | | | | |
|---|---|---|---|---|---|---|---|
| tation | | Treatment | 0 | 1 | 2 | 7 | 30 |
| A | Mean T_{lag} (m) | Unprimed | pre | pre | pre | pre | pre |
| | | Primed | pre | pre | pre | pre | pre |
| | Δ T_{lag} | | ND | ND | ND | ND | ND |

TABLE 8
Gₘₐₓ of SB3189 spores with and without heat priming.

| Fermen- | | | Days Since Priming | | | | |
|---|---|---|---|---|---|---|---|
| tation | | Treatment | 0 | 1 | 2 | 7 | 30 |
| A | Mean Gₘₐₓ (%) | Unprimed | 18 | 15 | 32 | 24 | 11 |
| | | Primed | 22 | 22 | 35 | 30 | 18 |
| | Δ Gₘₐₓ | | 24% | 44% | 11% | 26% | 64% |

### SB3002

Primed SB3002 demonstrated a significant reduction in T_{lag} compared to an unprimed control throughout the study (Table 9). As with SB3615, the magnitude of the improvement diminishes over time, but 30 days after treatment the primed spores were still germinating 29% sooner.

The Gₘₐₓ values for these spores also indicated that primed spores germinated with significantly greater efficiency compared to an untreated control (Table 10). The increase in Gₘₐₓ was no less than 66% throughout the experiment's duration.

TABLE 9
T_{lag} of SB3002 spores with and without heat priming.

| Fermen- | | | Days Since Priming | | | | |
|---|---|---|---|---|---|---|---|
| tation | | Treatment | 0 | 1 | 2 | 7 | 30 |
| A | Mean T_{lag} (m) | Unprimed | 139 | 96 | 64 | 79 | 163 |
| | | Primed | 31 | 44 | 38 | 51 | 116 |
| | Δ T_{lag} | | −78% | −54% | −41% | −35% | −29% |

TABLE 10
Gₘₐₓ of SB3002 spores with and without heat priming.

| Fermen- | | | Days Since Priming | | | | |
|---|---|---|---|---|---|---|---|
| tation | | Treatment | 0 | 1 | 2 | 7 | 30 |
| A | Mean Gₘₐₓ (%) | Unprimed | 15 | 24 | 33 | 29 | 11 |
| | | Primed | 62 | 45 | 60 | 48 | 20 |
| | Δ Gₘₐₓ | | 304% | 88% | 83% | 66% | 79% |

### SB3112

This particular strain is known to germinate poorly in advance of this study and it demonstrated that again. A T_{lag} value was calculated in only one instance (Table 11). It was only freshly heat primed spores that generated a measurable T_{lag}.

Even though germination response time for SB3112 was very slow, a response still did occur during the assay and a Gₘₐₓ was calculated (Table 12). Heat primed spores demonstrated significantly increased Gₘₐₓ at all time points after treatment compared to an untreated control.

TABLE 11
T_{lag} of SB3112 spores with and without heat priming.

| Fermen- | | | Days Since Priming | | | | |
|---|---|---|---|---|---|---|---|
| tation | | Treatment | 0 | 1 | 2 | 7 | 30 |
| A | Mean T_{lag} (m) | Unprimed | n/a | n/a | n/a | n/a | n/a |
| | | Primed | 100 | n/a | n/a | n/a | n/a |
| | Δ T_{lag} | | ND | ND | ND | ND | ND |

TABLE 12
Gₘₐₓ of SB3112 spores with and without heat priming.

| Fermen- | | | Days Since Priming | | | | |
|---|---|---|---|---|---|---|---|
| tation | | Treatment | 0 | 1 | 2 | 7 | 30 |
| A | Mean Gₘₐₓ (%) | Unprimed | 4 | 3 | 2 | 4 | 3 |
| | | Primed | 11 | 6 | 3 | 6 | 5 |
| | Δ Gₘₐₓ | | 167% | 111% | 29% | 42% | 67% |

### DISCUSSION

The only strain which demonstrated no benefit after heat activation (either shorter T_{lag} or higher Gₘₐₓ) was SB3086 (B. subtilis). In fact, under these conditions it appeared that heating led to a less efficient germination response. However, all the other strains tested did benefit from the heat activation in one way or another. SB3615 (B. amyloliquefaciens) and SB3002 (B. pumilis) both initiated germination sooner and had a higher proportion of spores that committed to germination when they are heat primed, and that benefit persisted at least for 30 days in cold storage. SB3130 (B. subtilis) also showed an improved germination response and commitment, but the effect was not as persistent compared to SB3615 and SB3002. It was no longer significant by the measures of this work after 7 days post-treatment. SB3189 (B. pumilis) unfortunately, prematurely germinated at some point before assay. For the heat primed sample, there was no discernible loss in viability, so germination most likely occurred in the space of time between priming and data collection. Furthermore, it was independent of heat priming, because it was observed in the unheated control as well. Regardless, the Gₘₐₓ of the strain was significantly increased when heat primed up to 30 days post-treatment. This result was notable because it was measurable above the background signal caused by premature germination. Generally, germination should not occur prematurely as a consequence of heat priming, because otherwise the spores will lose their natural resistances before they are in an environment that can support their viability. Finally, SB3112 (B. megaterium) had a slow germination response and a T_{lag} was undetectable in all but one case. The one instance of a measurable T_{lag} was by freshly primed spores. However, throughout the entire test period, the number of spores able to commit to germination remained significantly higher when heat activated. To our knowledge, this is the first instance where heat activation has been demonstrated to impact spore germination at a time point greater than 1 hour after treatment.

The impact of heat activation appeared to be strain dependent. This is best exemplified by SB3086 and SB3130. Both strains are B. subtilis, but the former appears to be hindered in its ability germinate after priming; the latter shows improvement. In addition, SB3615 and SB3002 had a similar outcome after priming despite their species difference.

The results indicated that the method of fermentation to generate the spores did not impact the efficacy of heat activation. For SB3086, the impact of heating was similarly detrimental to both batches of spores. For SB3615, both fermentation batches showed an improved speed and level of commitment for germination for the same duration post treatment.

This knowledge provides several potential benefits for industrial microbiology. Microbial products that are applied as spores may be limited initially in their ability to germinate. If the process happens too inefficiently then efficacy of the product suffers. The data show that a relatively simple, short, and sub-lethal heat priming can generate spores that remain dormant after treatment and up to 30 days after treatment. But once exposed to nutritive germinants, the primed spores can respond faster and more homogenously than what is normal. In some cases, the response time is cut in half and the magnitude of commitment is twice as high; it could have profound impact on how quickly a microbial product acts during application and what dose is required to generate the desired action.

### Example 2

### Varying Temperature and Duration of Heat Treatment of Bacillus Spores

Five strains of spores were heat primed at different temperatures and different durations to determine if either variable had an impact on the effectiveness of heat priming. The five strains tested encompassed members of three distinct species: Bacillus subtilis, Bacillus amyloliquefaciens, and Bacillus pumilus. Spores in an aqueous suspension were exposed to wet heat priming at either 60° C., 65° C., 70° C., 75° C., or 80° C. for durations of either 15, 30, 60, 120, or 240 m. A negative control was not subjected to heat priming. When not undergoing heat priming, all samples were stored at 4° C. until assayed for germination by the release of DPA.

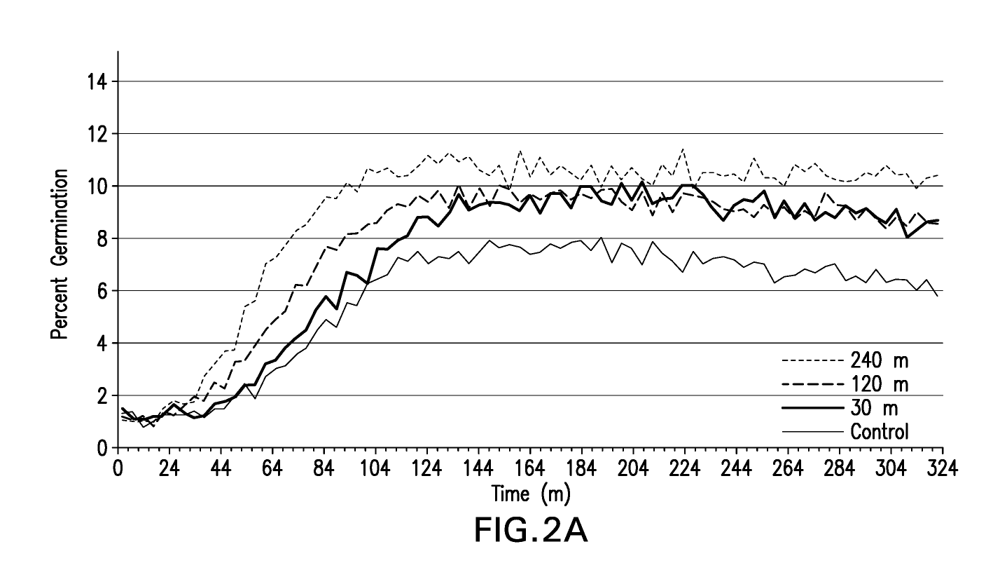

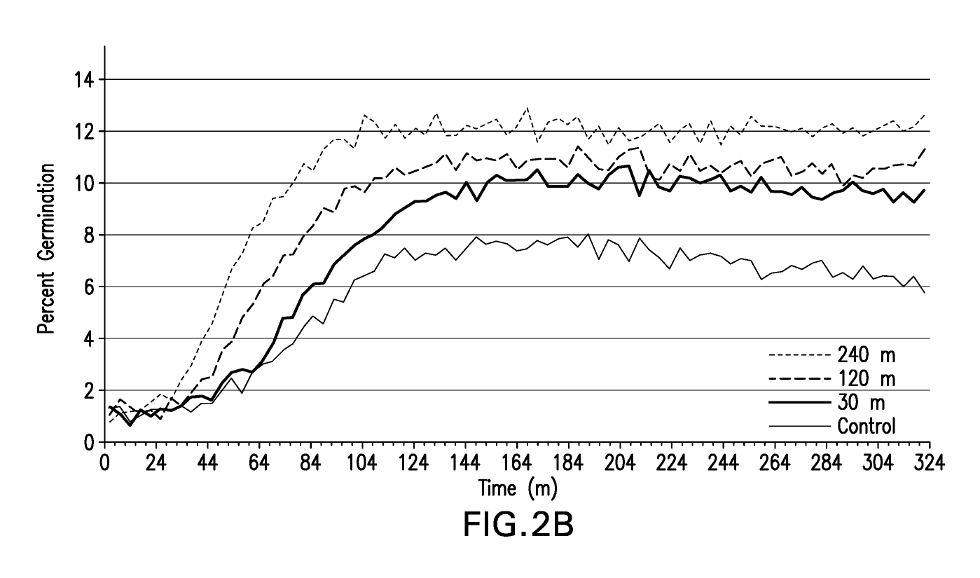

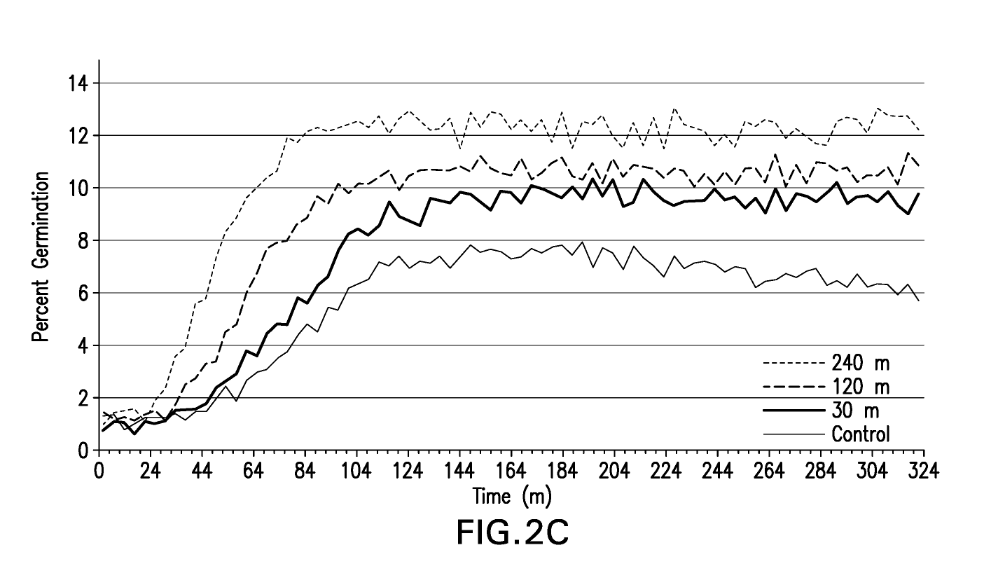

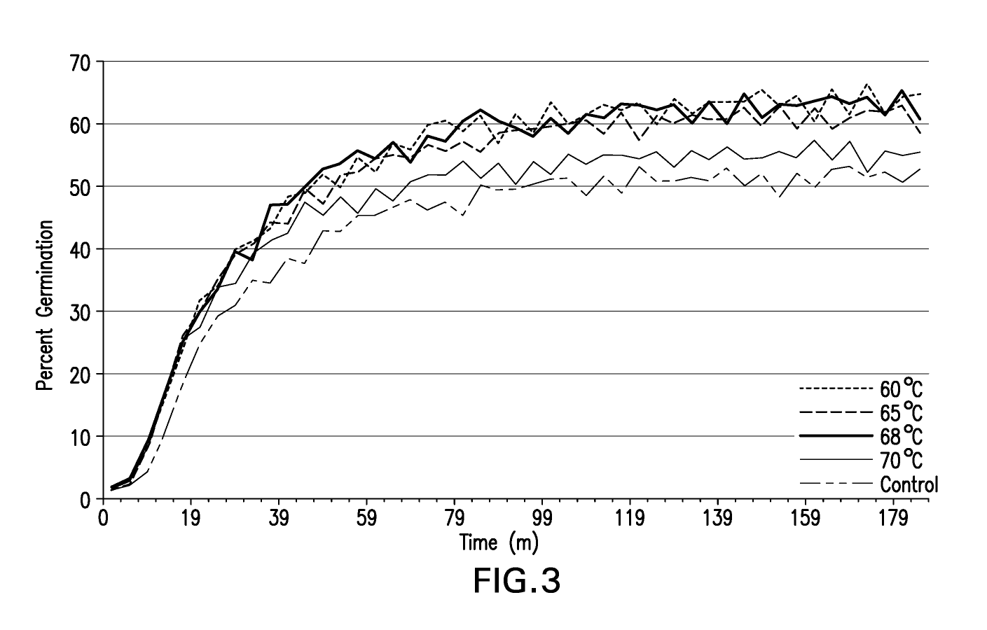

All strains responded favorably to heat priming in that exposure to wet heat for at least 15 minutes significantly improved germination kinetics by shortening T_{lag}, increasing Gₘₐₓ, or both, as demonstrated in FIGS. 2A-C and other examples. In addition, some strains, most notably B. subtilis, demonstrated trends where the germination kinetics continued to improve as the temperature and duration of heat priming increased (FIGS. 2A-C). This dose-dependent response towards temperature and/or duration was not demonstrated by every species. B. amyloliquefaciens, for example, derived a significant benefit from heat priming, but it was similar regardless of the temperature (60° C.-75° C.) or duration tested (30-240 m) (data not shown). B. pumilus, to the contrary, demonstrated a preference for 60° C.; increasing the heat priming temperature resulted in no significant change to the kinetics, and when ≥70° C. was used, the Gₘₐₓ reverted to what was seen with control spores (FIG. 3). Heat priming at temperatures ≥80° C. impacted the stability of the spores of all strains tested, because a significant number of the population prematurely germinated during or shortly after the heat priming (data not shown). Those spores that did not prematurely germinate still demonstrated improved germinant kinetics compared to a control.

Two other variables of the heat priming method demonstrated no impact on the results. They were the cool-down procedure after treatment and the storage temperature of the spores before and after treatment (data not shown). Cool-down was tested by quenching spores at 4° C. immediately after priming (rapid cool-down), incubation at 22° C. before storage, and incubation at 30° C. before storage. Storage conditions before and after priming were tested at 4° C. and 22° C. In all replicates and all strains tested, altering these variables demonstrated no deviation in the germination kinetics when compared to the appropriate control.

Heat priming can alter the germination kinetics of several Bacillus species. The factors for priming success are temperature and duration. The best temperature-duration combination to achieve shortest T_{lag} and highest Gₘₐₓ are strain specific. Those details are easily assessed by testing gradients of those two parameters side-by-side (FIGS. 2 and 3).

### Example 3

### Aquatic Animal Feed

In Southeast Asia, penaeid shrimp farms are being significantly damaged by outbreaks of a strain of Vibrio parahemolyticus. The disease is called “early mortality syndrome” and consequently the particular strain is commonly given the moniker “EMS.” The damage caused by the EMS strain is a major problem and attempts have been made to fine a Bacillus strain capable of inhibiting this pathogen.

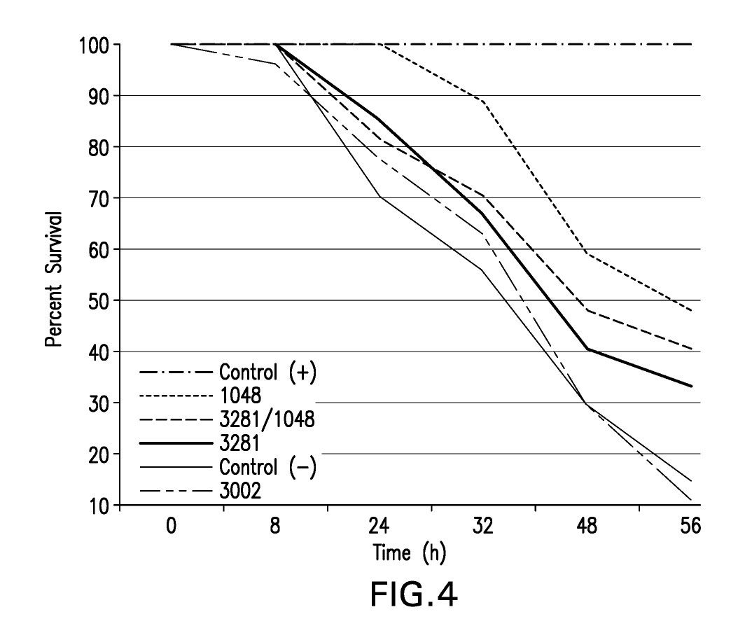

After a screening process, two candidate strains of Bacillus were identified based on their ability to inhibit EMS. The candidate strains were SB3281 and MF1048. Afterwards, a trial in shrimp was performed in the BSL2 shrimp lab at Virginia Tech (Blacksburg, Va.). In the trial, shrimp were given feed that was coated with spores of SB3281, MF1048, SB3002, a combination of SB3281/MF1048, or no spores (negative control). Shrimp were fed the corresponding feed mixture for 7 days before challenge with EMS (1E+9 cfu/g feed) and then mortality was assessed over time. For each treatment, 30 shrimp were tested split among three separate tanks. The results demonstrated that SB3281 and MF1048 spores, applied to the feed, did improve the shrimp survival when challenged with EMS (FIG. 4). These results were consistent with a previous independent trial using fewer shrimp (data not shown).

Despite these positive results, it was preferable to improve the shrimp survival more than what has currently been observed. It is likely spore germination is highly critical in this system, because of two facts: 1) the Bacillus are efficacious only in the vegetative form, and 2) the average gut transit time of penaeid shrimp ranges from 79-87 minutes and can be as short as 5 minutes. Consequently, the spores on feed must germinate in the gut to be present as vegetative cells and in a relatively short time frame. Therefore, we hypothesized that heat activated spores of SB3281 and MF1048 would improve shrimp survival during an EMS challenge due to faster and more homogenous spore germination in the gut of shrimp.

### Methods

### Endospore Preparation

Two strains of Bacillus were investigated for this study: SB3281 B. amyloliquefaciens and MF1048 Bacillus. sp. The endospore (spore) form of each organism was used in the study and was generated by Novozymes using a fermentation method which is a trade secret. Heat activation was performed by incubating the spores at 65° C. for 30 m. After fermentation and heat activation (if applicable), spores were stored at 4° C.

### Spore Application to Feed

Feed was coated with spores in the BSL2 shrimp lab at Virginia Tech (Blacksburg, Va.) in a rotating drum mixer via high pressure nozzle spray system and mixed thoroughly. After mixing, the concentration of spores on the feed ranged from 5E+07-6E+07 cfu/g of feed. Shrimp were fed the corresponding feed mixture for 7 days before challenge with EMS (1E+8 cfu/g feed) and then mortality was assessed over time. For each treatment, 30 shrimp were tested split evenly among three tanks. For heat activated spores, the time between activation and treatment on feed was 5 days. Mixed feed was stored at 4° C. until use.

### Results

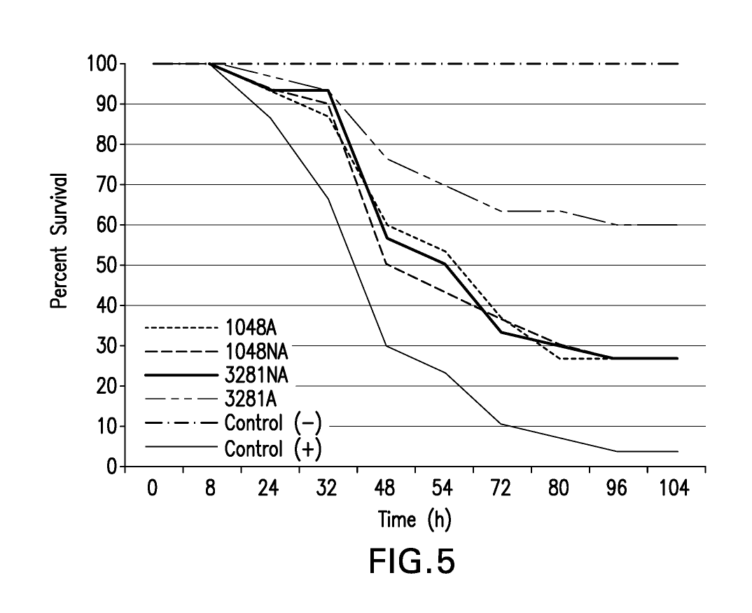

After feeding for 7 days, the shrimp were challenged with a lethal dose of EMS and the shrimp survival post-infection was assessed (FIG. 5). Shrimp fed MF1048 or SB3281 spores all demonstrated significant improvement in survival compared to a negative control receiving no spore treatment as determined by chi square analysis (p≤0.004). Furthermore, the shrimp treated with heat activated SB3281 (3281A) had the highest survival rate of all disease-challenged groups. This was significantly different as determined by chi square analysis (p=0.014).

### CONCLUSIONS

Penaeid shrimp given food coated with spores of SB3281 or MF1048 demonstrated significant improved survival when challenged with EMS V. parahemolyticus compared to a negative control where the food contained no spores. Shrimp survival was further improved when the SB3281 spores were heat activated prior to mixing with feed. After 104 h of infection with EMS, the survival of shrimp given heat activated SB3281 feed increased to 60% compared to 3% for shrimp given feed containing no spores.

We conclude that SB3281 spores germinate in the shrimp gut and that the vegetative cells inhibit the pathogenicity of EMS V. parahemolyticus. In addition, activation of the spores with sub-lethal heat primes their ability to germinate in the shrimp gut so that they germinate faster and more homogenously than unheated spores. As a consequence of this, more vegetative Bacillus can populate the shrimp gut before the spores are evacuated, where they can perform their anti-EMS activity.

It is known that heat activation can improve the speed and efficiency of spore germination, but it has been stated and disseminated anecdotally that those benefits are temporary with a window of effect of less than 1 day. Recall that the spores were activated 5 days before mixing with feed and then fed to the shrimp another 7 days before challenge with EMS. Thus we have demonstrated a long-term benefit of heat activation that persists for days to more than a week. This has not been shown anywhere before.

1. A stabilized bacterial spore composition, comprising:
(a) a carrier; and
(b) a bacterial spore population which has been treated with a sub-lethal heat treatment at 50-80° C. for more than 30 minutes followed by cooling to below 30° C.;
wherein the bacterial spore population exhibits improved germination after 24 hours compared to a non-treated, but otherwise identical, bacterial spore population.

2. The composition of claim 1, which is a substantially dry composition.

3. The composition of claim 1, wherein the bacterial spore population exhibits improved germination after 7 days compared to a non-treated bacterial spore population.

4. The composition of claim 1, wherein the heat treatment is carried out in an aqueous environment.

5. The composition of claim 1, wherein the heat treatment is carried out at 60-75° C. for 30-240 minutes followed by cooling to room temperature.

6. The composition of claim 1, wherein the bacterial spores are Bacillus spores.

7. The composition of claim 1, which is an animal feed composition and further comprises one or more animal feed additives.

8. The composition of claim 7, wherein the animal feed is an aquatic animal feed.

9. A method for providing vegetative bacterial cells of a bacterial spore population in the gut of an aquatic animal, comprising feeding the aquatic animal with the aquatic animal feed composition of claim 8.

10. The composition of claim 1, which further comprises a surfactant, a wetting agent, or a detergent builder.

11. A method for inhibiting or preventing malodor in a laundry washing machine, comprising contacting the laundry washing machine with the composition of claim 10.

12. The composition of claim 1, which further comprises one or more agriculturally beneficial ingredients.

13. A method for treating a plant or plant part, comprising contacting the plant or plant part with the composition of claim 12.

14. A method for preparing a stabilized bacterial spore composition, comprising the steps of:
(a) treating a bacterial spore population with a sub-lethal heat treatment at 50-80° C. for more than 30 minutes followed by cooling to below 30° C.;
(b) mixing the treated bacterial spore population with a carrier, and optionally one or more germinants; and
(c) storing the bacterial spore population for at least 24 hours before or after step (b);
wherein the bacterial spore population exhibits improved germination after 24 hours compared to a bacterial spore population which did not receive the treatment in (a).

15. The method of claim 14, wherein the bacterial spore population exhibits improved germination after 7 days compared to a bacterial spore population which did not receive the treatment in (a).

16. The composition of claim 1, further comprising
(c) one or more germinants.

17. The composition of claim 1, wherein the heat-treated bacterial spore population exhibits improved germination after 24 hours compared to an otherwise identical spore population that was heated for less than 30 minutes.

18. The composition of claim 1, wherein the heat-treated bacterial spore population exhibits improved germination for at least 60 days after the heat treatment, compared to the non-treated, but otherwise identical, bacterial spore population.

19. The composition of claim 1, wherein the heat-treated bacterial spore population germinates with a decreased T_{lag} or an increased Gₘₐₓ as compared to the non-treated, but otherwise identical, bacterial spore population.

20. A method, comprising:
heating a bacterial spore population to 50-80° C. for more than 30 minutes, followed by cooling to below 30° C.; and
germinating the heated bacterial spore population no sooner than 60 days after the heating,
wherein the heated bacterial spore population germinates with a decreased T_{lag} or an increased Gₘₐₓ as compared to a non-heat-treated, but otherwise identical, bacterial spore population.
